(19) United States
(12) Patent Application Publication
(10) Pub. No.: US 2020/0056234 A1
(43) Pub. Date: Feb. 20, 2020

(54) DNA METHYLATION SIGNATURES FOR DETERMINING A SURVIVAL PROBABILITY

(71) Applicant: Deutsches Krebsforschungszentrum, Heidelberg (DE)

(72) Inventors: Hermann Brenner, Wiesenbach (DE); Yan Zhang, Heidelberg (DE)

(21) Appl. No.: 16/487,301

(22) PCT Filed: Feb. 20, 2018
(86) PCT No.: PCT/EP2018/054106

§ 371 Date: Aug. 20, 2019

(30) Foreign Application Priority Data
Feb. 20, 2017 (EP) 17156961.9

(51) Int. Cl. C12Q 1/6876

(52) U.S. Cl. CPC C12Q 1/6876; C12Q 2600/154

(57) ABSTRACT

The present invention relates to a method for determining a survival probability of a subject comprising a) detecting the methylation status of at least two CpG sites selected from the list consisting of cg24704287, cg08362785, cg25983901, cg06126421, cg05575921, cg23665802, cg01612140, cg19572487, cg14975410, and cg10321156 in a sample of said subject and, b) based on the methylation status detected in step a), determining the survival probability of said subject. The present invention further relates to uses, data collections, kits, devices and methods related to the aforesaid method.

The present invention relates to a method for determining a survival probability of a subject comprising a) detecting the methylation status of at least two CpG sites selected from the list consisting of cg24704287, cg08362785, cg25983901, cg06126421, cg05575921, cg23665802, cg01612140, cg19572487, cg14975410, and cg10321156 in a sample of said subject and, b) based on the methylation status detected in step a), determining the survival probability of said subject. The present invention further relates to uses, data collections, kits, devices and methods related to the aforesaid method.

DNA methylation (DNAm), as the most widely studied form of epigenetic programming, has been revealed to be modulated by lifestyle and environmental factors (Dick, K. J. et al. DNA methylation and body-mass index: a genome-wide analysis. Lancet. 383, 1990-1998 (2014); Gao, X., Jia, M., Zhang, Y., Breitling, L. P. & Brenner, H. DNA methylation changes of whole blood cells in response to active smoking exposure in adults: a systematic review of DNA methylation studies. Clin Epigenetics. 7, 113 (2015)) and to be involved in onset and progression of complex diseases, including various forms of malignant diseases, cardiovascular diseases (CVD), metabolic diseases (e.g. diabetes), neuropsychiatric disorders, and autoimmune disorders (Feinberg, A. P. Genome-scale approaches to the epigenetics of common human disease. Virchows Arch. 456, 13-21 (2010); Zhong, J., Agha, G. & Baccarelli, A. A. The Role of DNA Methylation in Cardiovascular Risk and Disease: Methodological Aspects, Study Design, and Data Analysis for Epidemiological Studies. Circ Res. 118, 119-131 (2016); Chambers, J. C. et al. Epigenome-wide association of DNA methylation markers in peripheral blood from Indian Asians and Europeans with incident type 2 diabetes: a nested case-control study. Lancet Diabetes Endocrinol. 3, 526-534 (2015); Klengel, T., Pape, J., Binder, E. B. & Mehta, D. The role of DNA methylation in stress-related psychiatric disorders. Neuropharmacology. 80, 115-132 (2014); Dang, M. N., Buzzetti, R. & Pozzilli, P. Epigenetics in autoimmune diseases with focus on type 1 diabetes. Diabetes Metab Res Rev. 29, 8-18 (2013)). DNAm therefore could plausibly be associated with the excess mortality from specific diseases, and consequently with all-cause mortality. This was exemplified by the previous investigations on smoking-associated DNAm changes and their relationship with lung cancer incidence/mortality and mortality from any cause, cancer, and CVD (Zhang, Y. et al. Smoking-Associated DNA Methylation Biomarkers and Their Predictive Value for All-Cause and Cardiovascular Mortality. Environ Health Perspect. 124, 67-74 (2016); Zhang, Y. et al. F2RL3 methylation, lung cancer incidence and mortality. Int J Cancer. 137, 1739-1748 (2015); Zhang, Y. et al. F2RL3 methylation in blood DNA is a strong predictor of mortality. Int J Epidemiol. 43, 1215-1225 (2014)).

In addition, evidence has accumulated that the recently established ‘epigenetic clock’ (also known as DNAm age) based on age-associated DNAm changes, which presumably reflects individuals' biological age, is indicative for aging-related outcomes and longevity (Hannum, G. et al. Genome-wide methylation profiles reveal quantitative views of human aging rates. Mol Cell. 49, 359-367 (2013); Horvath, S. DNA methylation age of human tissues and cell types. Genome Biol. 14, R115 (2013); Marioni, R. E. et al. DNA methylation age of blood predicts all-cause mortality in later life. Genome Biol. 16, 25 (2015); Breitling, L. P. et al. Frailty is associated with the epigenetic clock but not with telomere length in a German cohort. Clin Epigenetics. 8, 21 (2016)). Following the first study reporting an association of DNAm age with all-cause mortality by Marioni et al., the association was consistently demonstrated in various longitudinal studies (Christiansen, L. et al. DNA methylation age is associated with mortality in a longitudinal Danish twin study. Aging Cell. 15, 149-154 (2016); Marioni, R. E. et al. The epigenetic clock and telomere length are independently associated with chronological age and mortality. Int J Epidemiol. 45, 424-432 (2016)), for individual age-associated CpGs (Lin, Q. et al. DNA methylation levels at individual age-associated CpG sites can be indicative for life expectancy. Aging (Albany N.Y.). 8, 394-401 (2016)), and also for newly identified age-associated CpGs (Moore, A. Z. et al. Change in Epigenome-Wide DNA Methylation Over 9 Years and Subsequent Mortality: Results From the InCHIANTI Study. J Gerontol A Biol Sci Med Sci. 71,1029-1035 (2016)). On the other hand, several epigenome-wide association studies (EWASs) have pointed out that DNAm involved in aging-related phenotypes are largely distinct from the established age-associated DNAm (Bell, J. T. et al. Epigenome-wide scans identify differentially methylated regions for age and age-related phenotypes in a healthy ageing population. PLoS Genet. 8, e1002629 (2012); Marttila, S. et al. Ageing-associated changes in the human DNA methylome: genomic locations and effects on gene expression. BMC Genomics. 16, 179 (2015); Jung, M. & Pfeifer, G. P. Aging and DNA methylation. BMC Biol. 13, 7 (2015)).

There is, thus, a need in the art for improved DNAm markers for determining an overall survival probability of an individual. This problem is solved by the means and methods disclosed herein.

Accordingly, the present invention relates to a method for determining a survival probability of a subject comprising

a) detecting the methylation status of at least two CpG sites selected from the CpG sites of Table 1 in a sample of said subject and,

b) based on the methylation status detected in step a), determining the survival probability of said subject.

As used in the following, the terms “have”, “comprise” or “include” or any arbitrary grammatical variations thereof are used in a non-exclusive way. Thus, these terms may both refer to a situation in which, besides the feature introduced by these terms, no further features are present in the entity described in this context and to a situation in which one or more further features are present. As an example, the expressions “A has B”, “A comprises B” and “A includes B” may both refer to a situation in which, besides B, no other element is present in A (i.e. a situation in which A solely and exclusively consists of B) and to a situation in which, besides B, one or more further elements are present in entity A, such as element C, elements C and D or even further elements.

Further, as used in the following, the terms “preferably”, “more preferably”, “most preferably”, “particularly”, “more particularly”, “specifically”, “more specifically” or similar terms are used in conjunction with optional features, without restricting further possibilities. Thus, features introduced by these terms are optional features and are not intended to restrict the scope of the claims in any way. The invention may, as the skilled person will recognize, be performed by using alternative features. Similarly, features introduced by “in an embodiment of the invention” or similar expressions are intended to be optional features, without any restriction regarding further embodiments of the invention, without any restrictions regarding the scope of the invention and without any restriction regarding the possibility of combining the features introduced in such way with other optional or non-optional features of the invention. Moreover, if not otherwise indicated, the term “about” relates to the indicated value with the commonly accepted technical precision in the relevant field, preferably relates to the indicated value±20%, more preferably±10%, most preferably±5%.

The method for determining a survival probability of the present invention, preferably, is an in vitro method. Moreover, it may comprise steps in addition to those explicitly mentioned above. For example, further steps may relate, e.g., to obtaining a sample for step a), deriving recommendations for further proceeding from the result of step b), and/or further steps as specified herein below. Moreover, one or more of said steps may be performed by automated equipment.

As used herein, the term “survival probability” relates to the probability that a subject will die within a certain period of time, wherein said period of time, preferably is at most 20 years, more preferably at most 17 years, more preferably at most 15 years, even more preferably at most 13 years, most preferably at most 10 years. Thus, preferably, the survival probability may be a favorable survival probability, i.e. a survival probability indicating a low probability for dying within one of the aforesaid time frames. Preferably, the survival probability for the aforesaid time frames in case a favorable survival probability is determined is at least 0.85, more preferably at least 0.9, most preferably at least 0.95. Also preferably, the survival probability may be an unfavorable survival probability, i.e. a survival probability indicating a decreased probability for surviving one of the aforesaid time frames. Preferably, the survival probability for the aforesaid time frames in case an unfavorable survival probability is determined is at most 0.85, more preferably at most 0.8, even more preferably at most 0.7, still more preferably at most 0.6, most preferably at most 0.5. In accordance with the above, “determining a survival probability” of a subject, as used herein, relates to determining the probability according to which the subject will die within one of the aforesaid time frames. Preferably, said probability is an overall mortality risk; in an embodiment, said probability is not a risk to die from a specific disease.

The method of the present invention, preferably, does not provide an indication that a subject is, at the time of assessment, afflicted with disease. Thus, in an embodiment, determining a survival probability is not diagnosing a specific disease, more preferably is not diagnosing disease. Thus, preferably, the method for determining a survival probability is not required to be performed by a medical practitioner, more preferably is not performed by a medical practitioner. Preferably, the result of the method of the present invention is not a diagnosis of disease, in particular not diagnosis of a disease which would require or be amenable to medical treatment at the time of assessment. As will be understood by the skilled person, in case an unfavorable survival probability is determined according to the method of the present invention, the subject may decide to or be recommended to perform life-style changes in order to improve its survival probability. In an embodiment, detecting an unfavorable survival probability, preferably, provides an indication that a subject has an increased probability to, preferably within the time frames as specified above, become afflicted with disease, preferably at least on of the diseases as specified herein. In a further embodiment, detecting an unfavorable survival probability, preferably, provides an indication that a subject has an increased probability to, preferably within the time frames as specified above, become afflicted with and die from disease, preferably at least on of the diseases as specified herein.

The term “subject” as used herein relates to an animal, preferably a mammal, and, more preferably, a human. Preferably, the subject according to the present invention is a subject of at least 40 years of age, more preferably at least 50 years of age, even more preferably at least 60 years of age, most preferably at least 65 years of age. Preferably, the subject is apparently healthy, i.e. has not been diagnosed with a disease requiring treatment at the time the method for determining a survival probability is performed. In an embodiment, the subject suffers from at least one of hypertension, diabetes, cardiovascular disease, and cancer at the time the method for determining a survival probability is performed. Preferably, the subject suffers from hypertension at the time the method for determining a survival probability is performed. As used herein, a subject is considered to suffer from hypertension if a systolic blood pressure ≥140 mmHg and/or a diastolic blood pressure ≥90 mmHg is diagnosed.

The term “sample”, as used herein, refers to a cell-comprising sample of a body fluid, to a sample of separated cells or to a sample from a tissue or an organ of the subject. Samples of body fluids can be obtained by well known techniques and include, preferably, samples of blood, plasma, serum, or urine. Tissue or organ samples may be obtained from any tissue or organ by, e.g., biopsy. Separated cells may be obtained from the body fluids or the tissues or organs by separating techniques such as centrifugation or cell sorting. Preferably, cell-, tissue- or organ samples are obtained from peripheral tissues. Preferably, the sample is a sample comprising blood cells, more preferably a blood product sample, e.g. a sample of whole blood or of a buffy coat. Blood samples can be obtained by well-known methods, in particular by arterial or venous puncture, and/or puncture of the skin.

The terms “CpG” and “CpG site” are known to the skilled person. Preferably, the terms relate to a site in DNA, preferably chromosomal DNA of a subject, having the nucleotide sequence 5′-CG-3′. As is also known to the skilled person, CpG sites can be methylated by DNA methyltransferases at the cytosine residue to yield a 5-methylcytosine residue, and methylation at a specific CpG site may be inherited or may be a de novo methylation acquired during life time of the subject. The CpG sites as referred to herein are those of Table 1. The CpG site locations indicated in Table 1 refer to the positions in the human reference genome GRCh37 as provided by the Genome Reference Consortium (www.ncbi.nlm.nih.gov/grc) on 2009 Feb. 27. This assembly is also referred to as hg19.

TABLE 1
CpG sites of the invention; positions on human chromosome and
nucleotide number of the CpG sites refer to the human genome
sequence assembly GRCh37/hg19.

| CpG site | Chr. position (GRCh37/hg19) |
|---|---|
| cg24704287 | 19p13.13 (chr19: 13951482) |
| cg08362785 | 22q13.1 (chr22: 40814879) |
| cg25983901 | 7p12.3 (chr7: 46972700) |
| cg06126421 | 6p21.33 (chr6: 30720081) |
| cg05575921 | 5p15.33 (chr5: 373378) |
| cg23665802 | 13q31.3 (chr13: 92002338) |
| cg01612140 | 6q14.1 (chr6: 78166437) |
| cg19572487 | 17q21.2 (chr17: 38476025) |
| cg14975410 | 3q26.31 (chr3: 171180070) |
| cg10321156 | 11q13.1 (chr11: 63687223) |
| cg03725309 | 1p13.3 (chr1: 109757585) |
| cg25763716 | 1p21.2 (chr1: 101184304) |
| cg13854219 | 1p21.2 (chr1: 101757037) |
| cg25189904 | 1p31.3 (chr1: 68299493) |
| cg15459165 | 1p35.2 (chr1: 31223850) |
| cg19266329 | 1q21.1 (chr1: 145456128) |
| cg24397007 | 2p23.2 (chr2: 28619095) |
| cg23079012 | 2p25.1 (chr2: 8343711) |
| cg27241845 | 2q37.1 (chr2: 233250371) |
| cg06905155 | 2q37.3 (chr2: 240723946) |
| cg16503724 | 3p24.3 (chr3: 17130667) |
| cg19859270 | 3q11.2 (chr3: 98251295) |
| cg02657160 | 3q12.1 (chr3: 98311063) |
| cg14855367 | 3q28 (chr3: 191048309) |
| cg14817490 | 5p15.33 (chr5: 392920) |
| cg21161138 | 5p15.33 (chr5: 399361) |
| cg12513616 | 5q35.3 (chr5: 177370977) |
| cg20732076 | 6p21.1 (chr6: 42335232) |
| cg25285720 | 6p21.32 (chr6: 32919434) |
| cg15342087 | 6p21.33 (chr6: 30720210) |
| cg12510708 | 7p15.2 (chr7: 26193806) |
| cg26286961 | 8p21.3 (chr8: 19460209) |
| cg00285394 | 8q24.13 (chr8: 126011954) |
| cg01140244 | 10q26.3 (chr10: 134498960) |
| cg23190089 | 11p15.4 (chr11: 2920209) |
| cg07123182 | 11p15.5 (chr11: 2722391) |
| cg26963277 | 11p15.5 (chr11: 2722408) |
| cg18550212 | 11q13.1 (chr11: 63435428) |
| cg25193885 | 11q13.3 (chr11: 70328867) |
| cg07986378 | 12p13.2 (chr12: 11898285) |
| cg04987734 | 14q32.32 (chr14: 103415874) |
| cg19459791 | 15q22.31 (chr15: 65363023) |
| cg00310412 | 15q24.1 (chr15: 74724919) |
| cg26709988 | 16q24.1 (chr16: 84860919) |
| cg23842572 | 17p11.2 (chr17: 17030253) |
| cg01572694 | 17q21.32 (chr17: 46657555) |
| cg08546016 | 17q25.1 (chr17: 72776239) |
| cg18181703 | 17q25.3 (chr17: 76354622) |
| cg03636183 | 19p13.11 (chr19: 17000586) |
| cg11341610 | 19p13.2 (chr19: 13050932) |
| cg14085840 | 19q13.2 (chr19: 40939429) |
| cg26470501 | 19q13.32 (chr19: 45252955) |
| cg05492306 | 19q13.32 (chr19: 45927594) |
| cg25607249 | 19q13.32 (chr19: 47288040) |
| cg01406381 | 19q13.32 (chr19: 47288263) |
| cg07626482 | 19q13.32 (chr19: 47289503) |
| cg03707168 | 19q13.33 (chr19: 49379127) |
| cg25491402 | 21q22.3 (chr21: 44101491) |

Preferably, the CpG sites analyzed according to the method of the present invention comprise sites selected from list consisting of cg24704287, cg08362785, cg25983901, cg06126421, cg05575921, cg23665802, cg01612140, cg19572487, cg14975410, and cg10321156, i.e. from the first ten CpG sites of Table 1. More preferably, the CpG sites analyzed according to the method of the present invention comprise sites selected from CpG sites of the list consisting of cg24704287, cg08362785, cg25983901, cg06126421, cg05575921, and cg23665802, i.e. from the first six CpG sites of Table 1.

As used herein, the term “methylation status” relates to a state of a specific CpG site in a cell being methylated or not, more preferably relates to the extent to which a specific CpG site is methylated in a population of cells, or not. As is understood by the skilled person, in a diploid cell, there are four occurrences of a specific CpG site, i.e. two alleles, with each allele comprising the two strands of DNA making up double-stranded DNA; thus, the methylation status of a single CpG site may be all four CpGs non-methylated; one CpG methylated; two, three, or four CpGs methylated. Also, in a population of cells, in particular in a mixed population of cells, the methylation status of a CpG site is not necessarily identical for all cells of said population. Thus, preferably, the methylation status is detected as the number of cells comprising a specific CpG site in methylated form in a given number of cells; or is detected as the number of methylated forms of a specific CpG site detected in a given number of cells. Preferably, the methylation status of at least 10, more preferably at least 25, most preferably at least 100 cells is detected in such case. More preferably, the methylation status is detected as a relative methylations status, e.g. in comparison to a population of a corresponding cells population obtained from one or more apparently healthy subjects. Most preferably, the methylation status is detected as a ratio of the number of individual CpG sites at a given position found to be methylated to the total number of individual CpG sites at said given position analyzed, which is known as the methylation beta value, or as a figure derivable therefrom by standard mathematical operations, e.g. the methylation M value (methylated/unmethylated) which can be calculated from Beta=2^{M}/(2^{M}+1); thus M=log2[Beta/(1−Beta)]. Thus, the methylation status of a CpG site in a population of cells, preferably, is the average degree of methylation of said CpG site in a population of at least 10, preferably at least 25, more preferably at least 100 cells. In an embodiment, the methylation status may also be expressed as a ratio of the number of individual CpG sites at a given position found to be unmethylated to the total number of individual CpG sites at said given position analyzed, i.e. as a non-methylation status. More preferably, the methylation status is expressed as a ratio of the number of individual CpG sites at a given position found to be methylated to the total number of individual CpG sites at said given position analyzed.

Methods for determining the methylation status of a CpG site are known in the art. Preferably, the method comprises isolating genomic DNA from said sample, preferably from cells comprised in said sample. Preferably, the method comprises contacting said DNA with a methylation-sensitive restriction enzyme having a nucleic acid sequence comprising the sequence 5′-CG-3′ as a recognition sequence; preferably, the method further comprises contacting a further aliquot of said DNA with a corresponding non-methylation-sensitive restriction enzyme having the same nucleic acid sequence comprising the sequence 5′-CG-3′ as a recognition sequence. More preferably, the method comprises treating said DNA, before of after isolation, with a bisulfite, preferably sodium bisulfite. Preferably, the method further comprises annealing an oligonucleotide specifically annealing to a sequence immediately upstream of said CpG site and comprising a 3′-terminal sequence 5′-CG-3′ and/or an oligonucleotide specifically annealing to a sequence immediately upstream of said CpG site and comprising a 3′-terminal sequence 5′-CA-3′ to said genomic DNA, preferably to said bisulfite-treated genomic DNA, per CpG site. Preferably, the method further comprises performing a one-nucleotide extension reaction after said annealing in such case. Also preferably, the method comprises annealing per CpG site an oligonucleotide specifically annealing to a sequence immediately upstream of said CpG site and having a C as the terminal nucleotide, and performing pyrosequencing using said oligonucleotide as a sequencing primer.

According to the method for determining a survival probability, the methylation status of at least two CpG sites selected from Table 1 is determined. As is understood by the skilled person, accuracy of prediction may be increased by determining the methylation status of an increased number of CpG sites; thus, preferably, the methylation status of from three to all, more preferably of from five to 50, even more preferably of from 6 to 25, most preferably of from 7 to 12 CpG sites of Table 1 is determined. More preferably, the methylation status of at least three, preferably at least four, more preferably at least five, most preferably at least six CpG sites selected from cg24704287, cg08362785, cg25983901, cg06126421, cg05575921, cg23665802, cg01612140, cg19572487, cg14975410, and cg10321156 is detected. Most preferably, the methylation status of all ten aforesaid methylation sites is determined. Also preferably, the methylation status of at least three, preferably at least four, more preferably at least five, most preferably all six CpG sites selected from cg24704287, cg08362785, cg25983901, cg06126421, cg05575921, and cg23665802 is detected.

Preferably, an unfavorable survival probability is determined if a methylation status deviating from a reference is detected, preferably is detected for at least two, more preferably at least three, even more preferably at least four, most preferably at least five CpG sites. Thus, preferably, the method for determining a survival probability comprises comparing the methylation status determined for a CpG site in a sample to a reference. Thus, preferably, the method comprises further step a1) comparing the methylation status of said at least two CpG sites of step a) to references; and wherein in step b) the determining is based on the comparison of step a1). As used herein, the term “reference” relates to a reference value or a reference range, preferably derived from a population of subjects, preferably a population of apparently healthy subjects as specified herein above. Also preferably, reference values or reference ranges are predetermined references, which may, e.g. be provided in the form of a database, a list, or the like.

Methods for determining a, preferably significant, more preferably statistically significant, deviation of a methylation status from a reference are known to the skilled person; preferably, a value and a reference value are determined to be essentially identical if the difference between two values is, preferably, not significant and shall be characterized in that the value is within at least the interval between 1st and 99th percentile, 5th and 95th percentile, 10th and 90th percentile, 20th and 80th percentile, 30th and 70th percentile, 40th and 60th percentile of the reference value, preferably, the 50th, 60th, 70th, 80th, 90th or 95th percentile of the reference value. Statistical test for determining whether two amounts are essentially identical are well known in the art and are also described elsewhere herein. Conversely, an observed difference for two values, on the other hand, shall preferably be statistically significant. A difference in value is, preferably, significant outside of the interval between 45th and 55th percentile, 40th and 60th percentile, 30th and 70th percentile, 20th and 80th percentile, 10th and 90th percentile, 5th and 95th percentile, 1st and 99th percentile of the reference value. Preferably, in case a decrease in the value of the methylation status is indicative of an unfavorable survival probability, such as in CpG sites cg24704287, cg25983901, cg06126421, cg05575921, cg23665802, cg01612140, cg19572487, cg14975410, and cg10321156, a methylation status value lying in the first (i.e. lowest) quartile of reference values of a population of subjects is considered to be significantly different. Also preferably, in case an increase in the value of the methylation status is indicative of an unfavorable survival probability, such as in CpG site cg08362785, a methylation status value lying in the last (i.e. highest) quartile of reference values of a population of subjects is considered to be significantly different. More preferably, the reference value or reference range is a cut-off value of an average degree of methylation of ≤0.34 for cg01612140; ≤0.78 for cg05575921; ≤0.60 for cg06126421; ≥0.67 for cg08362785; ≤0.39 for cg10321156; ≤0.45 for cg14975410; ≤0.49 for cg19572487; ≤0.30 for cg23665802; ≤0.31 for cg24704287; and/or ≤0.49 for cg25983901. Whether a difference is statistically significant can be determined without further ado by the person skilled in the art using various well known statistic evaluation tools, e.g., determination of confidence intervals, p-value determination, Student's t-test, Mann-Whitney test etc. Details are found in Dowdy and Wearden, Statistics for Research, John Wiley & Sons, New York 1983. Preferred confidence intervals are at least 90%, at least 95%, at least 97%, at least 98% or at least 99%. The p-values are, preferably, 0.1, 0.05, 0.01, 0.005, or 0.0001. Preferably, the probability envisaged by the present invention allows that the determination will be correct for at least 60%, at least 70%, at least 80%, or at least 90% of the subjects of a given cohort or population. Further methods of evaluating statistical significance of differences in methylation ae described herein below in the Examples.

As indicated above, at least two CpG sites are evaluated according to the present invention. As is understood by the skilled person, the value detected for a specific CpG site is compared to a corresponding CpG site, i.e. to a reference value pertaining to the CpG site having the same position in the genome. Thus, in case e.g. the average degree of methylation is determined for the ten first CpG sites of Table 1, each of these values is compared to a corresponding reference value, respectively. As is also understood by the skilled person, values are compared to corresponding values, i.e. average degree of methylation values are compared to average degree of methylation values, numbers of cells comprising the CpG site in methylated form are compared to numbers of cells comprising the CpG site in methylated form, and the like. Preferably, an unfavorable heath state is determined if at least one of said CpG sites deviates, preferably significantly deviates, from the reference value. More preferably, an unfavorable survival probability is determined if a methylation status deviating, preferably significantly deviating, from the reference is detected for at least two, more preferably at least three, even more preferably at least four, still more preferably at least five, most preferably more than five CpG sites. Most preferably, an unfavorable survival probability is determined if a methylation status deviating, preferably significantly deviating, from the reference is detected for at least two, more preferably at least three, even more preferably at least four, still more preferably at least five, most preferably more than five CpG sites selected from the first ten CpG sites of Table 1. Preferably, determining a survival probability comprises calculating a score from the values detected for the CpG sites, which may, preferably, include a weighting of the CpG sites analyzed. Preferably, said score is calculated as a continuous risk score according to: cg01612140*(−0.38253)+cg05575921*(−0.92224)+cg06126421*(−1.70129)+cg08362785*(2.71749)+cg10321156*(−0.02073)+cg14975410*(−0.04156)+cg19572487*(−0.28069)+cg23665802*(−0.89440)+cg24704287*(−2.98637)+cg25983901*(−1.80325). Preferably, references and/or evaluation algorithms are stored on a suitable data storage medium, preferably in the form of a database and are, thus, also available for future assessments.

Advantageously, it was found in the work underlying the present invention that the methylation status of the indicated CpG sites, in particular the first ten CpG sites of Table 1, is an independent indicator of the overall mortality risk of a subject, independent of potentially prevalent underlying disease and independent of the biological age of the subject.

The definitions made above apply mutatis mutandis to the following. Additional definitions and explanations made further below also apply for all embodiments described in this specification mutatis mutandis

The present invention further relates to a method for patient monitoring comprising the steps of the method for determining a survival probability and providing close monitoring and/or lifestyle recommendations in case an unfavorable survival probability and/or an increased mortality risk is detected.

The method for patient monitoring of the present invention, preferably, is an in vitro method. Moreover, it may comprise additional steps, e.g. as specified herein above. As is understood from the above, the method for patient monitoring, preferably is not a method of diagnosing disease. Preferably, the method for patient monitoring is a supportive measure aiding the medical practitioner in deciding on which tests to perform to establish a diagnosis. E.g. detecting an unfavorable survival probability in a subject, or having detected an unfavorable survival probability in said subject in the past, preferably in a time frame as specified herein above, may give reason to perform additional tests in a subject having symptoms, in particular showing at least one symptom of at least one of hypertension, diabetes, cardiovascular disease, and cancer, preferably of cardiovascular disease.

The term “close monitoring”, as used herein, relates to performing follow-up examinations at a higher frequency as would be performed on a normal subject. Life-style recommendations for improving the survival probability of a subject are, in principle, known in the art and, preferably, depend on specific risk factors of the subject. Preferably, life-style recommendations relate to increasing exercise and/or eating habits, decreasing body fat, cessation of alcohol consumption and/or smoking, improvement of sleeping habits, in particular sleep/wake cycles, and the like.

The present invention further relates to a use of the methylation status of genomic DNA or means for the determination thereof in a sample of a subject for determining a survival probability of said subject, preferably for predicting the mortality risk of said subject.

The present invention further relates to a data collection, preferably comprised on a data carrier, comprising the positions of at least two, preferably at least three, more preferably at least four, even more preferably at least five, most preferably at least six CpG sites selected from cg24704287, cg08362785, cg25983901, cg06126421, cg05575921, cg23665802, cg01612140, cg19572487, cg14975410, and cg10321156; preferably of from three to all, more preferably of from five to 50, even more preferably of from 6 to 25, most preferably of from 7 to 12 CpG sites selected from Table 1.

Moreover, the present invention relates to a kit comprising means for determining the methylation status of at least two CpG sites selected from the CpG sites of Table 1, preferably selected from cg24704287, cg08362785, cg25983901, cg06126421, cg05575921, cg23665802, cg01612140, cg19572487, cg14975410, and cg10321156, and a data collection according to the present invention.

The term “kit”, as used herein, refers to a collection of the aforementioned compounds, means or reagents of the present invention which may or may not be packaged together. The components of the kit may be comprised by separate housings (i.e. as a kit of separate parts), or two or more components may be provided in a single housing. Moreover, it is to be understood that the kit of the present invention, preferably, is to be used for practicing the methods referred to herein above. It is, preferably, envisaged that components are provided in a ready-to-use manner for practicing the methods referred to above. In an embodiment, all or some of the chemical compounds of the kit are provided in dried, such as in lyophilized form, wherein the component is reconstituted using a liquid such as water or an aqueous buffered solution. In an embodiment, all or some of said compounds are provided in concentrated liquid form wherein the concentrated component is diluted using a liquid such as an aqueous buffered solution. In an embodiment, all or some of said compounds are provided in frozen form wherein the components are thawed prior to use. In an embodiments all or some of said compounds are in a liquid ready-to-use form. Further, the kit, in an embodiment, contains instructions for carrying out said methods and, if applicable, said reconstitution of dried reagents. The instructions and data collection can be provided in paper- or electronic form, e.g. by a user's manual or as a database. In addition, the manual may comprise instructions for interpreting the results obtained when carrying out the aforementioned methods using the kit of the present invention.

The present also relates to a device comprising an analysis unit comprising means for determining the methylation status of at least two CpG sites selected from the CpG sites of Table 1, preferably selected from cg24704287, cg08362785, cg25983901, cg06126421, cg05575921, cg23665802, cg01612140, cg19572487, cg14975410, and cg10321156, and an evaluation unit comprising a data collection according to the present invention.

The term “device”, as used herein, relates to a system of means comprising at least the means described, operatively linked to each other as to allow the determination. How to link the means of the device in an operating manner will depend on the type of means included into the device. In an embodiment, the means are comprised by a single device. However, it is also contemplated that the means of the current invention, e.g. the analysis unit and the evaluation unit, in an embodiment, may appear as separate devices and are, preferably, packaged together as a kit. The person skilled in the art will realize how to link the means without further ado. Preferred devices are those which can be applied without the particular knowledge of a specialized technician. Preferably, the device is adapted to include an additional feature as described herein. Preferably, the device further comprises (i) a display unit displaying a survival probability determined or raw data related thereto; and/or comprises (ii) a memory unit storing methylation status data and/or reference data determined. In a further embodiment, the device further comprises an output unit operatively linked at least to the evaluation unit, which output unit may be a simple signal generator such as a warning lamp or a device providing an audible signal, but may also be a display device or a printer.

The device comprises an analysis unit comprising means for determining a methylation status of at least two CpG sites. Typical means and methods for determining a methylation status are known in the art and exemplary means are described elsewhere herein, in particular in the examples. Preferably, the methylation status is determined as specified herein in the Examples.

In view of the above, the following embodiments are preferred:

1. A method for determining a survival probability of a subject comprising

a) detecting the methylation status of at least two CpG sites selected from the CpG sites of Table 1 in a sample of said subject and,

b) based on the methylation status detected in step a), determining the survival probability of said subject.

2. The method of claim 1, wherein the methylation status of from three to all, preferably of from five to 50, more preferably of from 6 to 25, most preferably of from 7 to 12 CpG sites of Table 1 is determined.

3. The method of claim 1 or 2, wherein said at least two CpG sites are selected from the list consisting of cg24704287, cg08362785, cg25983901, cg06126421, cg05575921, cg23665802, cg01612140, cg19572487, cg14975410, and cg10321156.

4. The method of claim 3, wherein the methylation status of at least three, preferably at least four, more preferably at least five, most preferably at least six of said CpG sites is detected.

5. The method of claim 3 or 4, wherein the methylation status of all ten methylation sites is determined.

6. The method of any one of claims 1 to 5, wherein said detecting the methylation status of a CpG site is detecting the average degree of methylation of said site from at least 10, preferably at least 25, more preferably at last 100 cells.

7. The method of any one of claims 1 to 6, wherein said method comprises further step a1) comparing the methylation status of said at least two CpG sites of step a) to references; and wherein in step b) the determining is based on the comparison of step a1).

8. The method of claim 7, wherein said reference is a reference value or a reference range.

9. The method of claim 7 or 8, wherein said reference is obtained from a population of subjects, preferably a population of apparently healthy subjects.

10. The method of any one of claims 7 to 9, wherein said reference value or reference range is an average degree of methylation of ≤0.34 for cg01612140; ≤0.78 for cg05575921; ≤0.60 for cg06126421; ≥0.67 for cg08362785; ≤0.39 for cg10321156; ≤0.45 for cg14975410; ≤0.49 for cg19572487; ≤0.30 for cg23665802; ≤0.31 for cg24704287; and/or ≤0.49 for cg25983901.

11. The method of any one of claims 1 to 10, wherein said sample is a sample comprising cells of said subject, preferably nucleate cells.

12. The method of any one of claims 1 to 11, wherein said sample is a bodily fluid sample, preferably is a blood sample or a sample of blood cells.

13. The method of any one of claims 1 to 12, wherein said subject is a human.

14. The method of any one of claims 1 to 13, wherein an unfavorable survival probability is determined if a methylation status deviating from the reference is detected, preferably is detected for at least two, more preferably at least three, even more preferably at least four, most preferably at least five CpG sites.

15. The method of any one of claims 1 to 14, wherein determining said survival probability comprises determining a mortality risk.

16. The method of claim 15, wherein said mortality risk is an overall mortality risk.

17. The method of claim 15 or 16, wherein said mortality risk not an indication-specific mortality risk.

18. The method of any one of claims 15 to 17, wherein a high mortality risk is determined if a methylation status deviating from the reference, preferably deviating as indicated in Table 1, is detected, preferably is detected for at least two, more preferably at least three, even more preferably at least four, most preferably at least five CpG sites is detected.

19. The method of any one of claims 1 to 18, wherein said method comprises isolating genomic DNA from said sample.

20. The method of claim 19, wherein said method comprises treating said genomic DNA with a bisulfite.

21. The method of claim 19 or 20, wherein said method comprises annealing an oligonucleotide specifically annealing to a sequence immediately upstream of said CpG site and comprising a 3′-terminal sequence 5′-CG-3′ and/or an oligonucleotide specifically annealing to a sequence immediately upstream of said CpG site and comprising a 3′-terminal sequence 5′-CA-3′ to said genomic DNA, preferably to said bisulfite-treated genomic DNA, per CpG site.

22. The method of claim 21, wherein said method comprises performing a one-nucleotide extension reaction after said annealing.

23. The method of any one of claims 1 to 22, wherein said method comprises annealing per CpG site an oligonucleotide specifically annealing to a sequence immediately upstream of said CpG site and having a C as the terminal nucleotide and performing pyrosequencing using said oligonucleotide as a sequencing primer.

24. The method of any one of claims 1 to 23, wherein said determining a survival probability is not diagnosing disease.

25. The method of any one of claims 1 to 24, wherein the CpG sites correlate with positions in the human genome as shown in Table 1.

26. A method for patient monitoring comprising the steps of the method according to any one of claims 1 to 25 and providing close monitoring and/or lifestyle recommendations in case an unfavorable survival probability and/or an increased mortality risk is detected.

27. Use of the methylation status of genomic DNA or means for the determination thereof in a sample of a subject for determining a survival probability of said subject, preferably for predicting the mortality risk of said subject.

28. The use of claim 27, wherein said use comprises detecting the methylation status of at least two CpG sites selected from the CpG sites of Table 1, preferably selected from cg01612140, cg05575921, cg06126421, cg08362785, cg10321156, cg14975410, cg19572487, cg23665802, cg24704287, and cg25983901.

29. A data collection, preferably comprised on a data carrier, comprising the positions of at least two, preferably at least three, more preferably at least four, even more preferably at least five, most preferably at least six CpG sites selected from cg24704287, cg08362785, cg25983901, cg06126421, cg05575921, cg23665802, cg01612140, cg19572487, cg14975410, and cg10321156; preferably of from three to all, more preferably of from five to 50, even more preferably of from 6 to 25, most preferably of from 7 to 12 CpG sites selected from Table 1.

30. The data collection of claim 29, further comprising reference values or reference ranges for the methylation status of said CpG sites.

31. A kit comprising means for determining the methylation status of at least two CpG sites selected from the CpG sites of Table 1, preferably selected from cg24704287, cg08362785, cg25983901, cg06126421, cg05575921, cg23665802, cg01612140, cg19572487, cg14975410, and cg10321156, and a data collection according to claim 29 or 30.

32. A device comprising an analysis unit comprising means for determining the methylation status of at least two CpG sites selected from the CpG sites of Table 1, preferably selected from cg24704287, cg08362785, cg25983901, cg06126421, cg05575921, cg23665802, cg01612140, cg19572487, cg14975410, and cg10321156, and an evaluation unit comprising a data collection according to claim 29 or 30.

All references cited in this specification are herewith incorporated by reference with respect to their entire disclosure content and the disclosure content specifically mentioned in this specification.

### FIGURE LEGENDS

FIG. 1: Flowchart of study design and data analysis

FIG. 2: Distribution of deceased across the 10 CpGs used to define the risk score in ESTHER [panel a; N=1000 (231 deaths)] and KORA study [panel b; N=1727 (61 deaths)].

FIG. 3: Kaplan-Meier estimates of survival by risk score in the ESTHER study (N=1000). a. Survival curves with respect to death from any causes; b. Survival curves with respect to death from cancer; c. Survival curves with respect to death from cardiovascular disease (CVD). Plog-rank was derived from log-rank test.

FIG. 4: Kaplan-Meier estimates of survival by risk score in the KORA study (N=1727). a. Survival curves with respect to death from any causes; b. Survival curves with respect to death from cancer; c. Survival curves with respect to death from cardiovascular disease (CVD). Plog-rank was derived from log-rank test.

FIG. 5: Dose-response relationships between continuous risk score and all-cause mortality. a. Dose-response curve in the ESTHER study [N=1,000 (231 deaths)]; b. Dose-response curve in the KORA study [N=1,727 (61 deaths)].

The following Examples shall merely illustrate the invention. They shall not be construed, whatsoever, to limit the scope of the invention.

### EXAMPLE 1

### Materials and Methods

Study Population and Data Collection

The EWAS and subsequent validation were conducted in the ESTHER study, an ongoing population-based cohort study conducted in Saarland, Germany. The ESTHER cohort, as previously described in detail (Schottker, B. et al. Strong associations of 25-hydroxyvitamin D concentrations with all-cause, cardiovascular, cancer, and respiratory disease mortality in a large cohort study. Am J Clin Nutr. 97, 782-793 (2013)), enrolled 9,949 older adults (age 50-75 years) by their general practitioners (GPs) during routine health check-ups between 2000 and 2002. The participants completed a standardized self-administered questionnaire and donated biological samples (blood, stool, urine) during baseline enrolment. Comprehensive medical data, such as the results of a physical assessment, medical diagnoses, and drug prescriptions were additionally obtained from the GPs. Deaths during follow-up were identified through record linkage with population registries in Saarland. Information on the major cause of death was obtained from death certificates provided by the local health authorities, and coded with ICD-10-codes. Deaths from CVD and malignant invasive cancers, respectively, were defined by ICD-10 codes 100-199 and C00-C97 (excluding non-melanoma skin cancer (C44)).

Genome-wide DNAm measurements were performed in the baseline blood samples of two subsets of the ESTHER participants. Subset-I (discovery panel) consists of participants from a case-cohort study nested within 2,499 ESTHER participants who were consecutively recruited between October 2000 and March 2001 and had sufficient DNA available. Of the 2,499 participants, the 406 who died during follow-up by March 2013 were the cases in the case-cohort design, and 548 were randomly selected as the subcohort irrespective of death status during follow-up. The sampling fraction was thus 548/2,499=22%. Subset-II (validation panel) consists of 1,000 ESTHER participants who were recruited between July and October 2000 and who were non-overlapping with the case-cohort samples, among whom 231 deaths were ascertained during follow-up.

Replication in an independent cohort was performed in the KORA F4 study, a population-based cohort consisting of 3,080 participants (age 32-81 years) recruited between 2006 and 2008 from the region of Augsburg, Southern Germany (Holle, R., Happich, M., Lowel, H., Wichmann, H. E. & Group, M. K. S. KORA—a research platform for population based health research. Gesundheitswesen. 67 Suppl 1, S19-25 (2005); Zeilinger, S. et al. Tobacco smoking leads to extensive genome-wide changes in DNA methylation. PLoS One. 8, e63812 (2013)). The vital status of KORA participants was ascertained through population registries inside and outside the study area in December 2011. Causes of death were determined according to death certificates from the Regional Health Department and coded with ICD-9. A random baseline sample consisting of 1,727 participants were selected for methylation analysis, among whom 61 participants died.

All ESTHER and KORA F4 participants provided written informed consent. The ESTHER study was approved by the ethics committees of the University of Heidelberg and of the state medical board of Saarland, Germany. The KORA F4 study was approved by the Ethics Committee of the Bavarian Medical Association.

Methylation Assessment

DNAm in whole blood was quantified using the Infinium HumanMethylation450K BeadChip (Illumina.Inc, San Diego, Calif., USA) in both ESTHER and KORA F4. Details of methylation analysis in the ESTHER study have been reported previously (Zhang, Y. et al. Smoking-Associated DNA Methylation Biomarkers and Their Predictive Value for All-Cause and Cardiovascular Mortality. Environ Health Perspect. 124, 67-74 (2016); Zhang, Y., Florath, I., Saum, K. U. & Brenner, H. Self-reported smoking, serum cotinine, and blood DNA methylation. Environ Res. 146, 395-403 (2016)). According to the manufacturer's protocol, data were normalized to internal controls provided by Illumina (Illumina normalization). In data pre-processing, probes with detection p-value >0.01, with missing values >10%, probes targeting the sex chromosomes, cross-reactive probes and polymorphic CpGs (Chen, Y. A. et al. Discovery of cross-reactive probes and polymorphic CpGs in the Illumina Infinium HumanMethylation450 microarray. Epigenetics. 8, 203-209 (2013)) were excluded, leaving 430,363 CpGs for genome-wide screening. In the KORA study, data were pre-processed following the pipeline of Lehne et al. (Lehne, B. et al. A coherent approach for analysis of the Illumina HumanMethylation450 BeadChip improves data quality and performance in epigenome-wide association studies. Genome Biol. 16, 37 (2015)), probes with detection p-value (1-p-value computed from the background model characterizing the probability that the target sequence signal was distinguishable from the negative controls)>0.01 and missing values>5% were removed, and quantile normalization was applied following stratification of the probe categories into 6 types, based on probe type and color channel, using the R package limma (Smyth, G. in Bioinformatics and Computational Biology Solutions Using R and Bioconductor (eds R. Gentleman et al.) 397-420 (Springer, 2005)). Leukocyte composition was estimated using Houseman et al.'s algorithms (Houseman, E. A. et al. DNA methylation arrays as surrogate measures of cell mixture distribution. BMC Bioinformatics. 13, 86 (2012)) in both studies.

Statistical Analysis

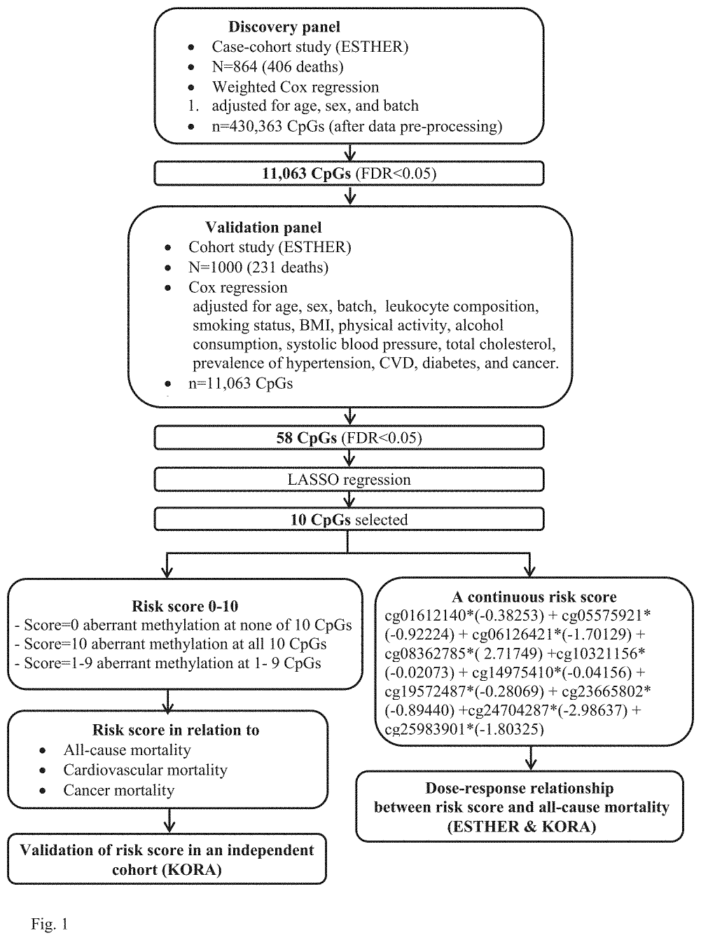

Discovery and validation of mortality-related CpGs. The ESTHER study populations were described separately in the discovery and validation panel with respect to major sociodemographic characteristics, lifestyle factors, and prevalent diseases at baseline. An epigenome-wide screening for mortality-related CpGs was first carried out in the case-cohort samples, using weighted Cox regression models that account for the case-cohort sampling design by Barlow-weighting (the inverse of the subcohort sampling fraction, 1/(548/2499))(Barlow, W. E. Robust variance estimation for the case-cohort design. Biometrics. 50, 1064-1072 (1994); Kulathinal, S., Karvanen, J., Saarela, O. & Kuulasmaa, K. Case-cohort design in practice—experiences from the MORGAM Project. Epidemiol Perspect Innov. 4, 15 (2007)). The models with methylation β-values as explanatory variables were adjusted for age, sex, and batch effects. After correcting for multiple testing using the Benjamini-Hochberg approach, CpGs that reached genome-wide significance (FDR <0.05) were entered into the validation phase, in which the associations with mortality were further analysed by multiple Cox regression adjusted for age, sex, batch effects, leukocyte composition, smoking status (never, former, and current smoker), body mass index (BMI, kg/m²), physical activity (inactive, low, medium/high), alcohol consumption (g per day), systolic blood pressure (mmHg), total cholesterol level (mg/dL), and prevalence of hypertension, CVD, diabetes, and cancer. CpGs with FDR<0.05 in the validation panel were deemed as mortality-related loci. A flowchart of study design and data analysis is shown in FIG. 1.

Associations of risk factors with mortality-related CpGs. To explore risk factors related to methylation associated with fatal endpoints, sociodemographic characteristics, lifestyle factors, and prevalent diseases at baseline were assessed in relation to the methylation levels of the identified CpGs using mixed linear regression models in the validation panel, with batch as random effect, methylation β-value as the dependent variable, and independent variables including age, sex, smoking status (never, former, and current smoker), BMI (underweight/normal weight, overweight, and obesity), physical activity (inactive, low, medium/high), alcohol consumption (g per day), and prevalent hypertension, diabetes, CVD, and cancer, again controlling for leukocyte composition. Multiple testing was again corrected for by the Benjamini-Hochberg approach (FDR<0.05).

Mortality risk score. To develop a DNAm-based mortality risk score, we applied the least absolute shrinkage and selection operator (LASSO) Cox regression (Tibshirani, R. The lasso method for variable selection in the Cox model. Stat Med. 16, 385-395 (1997)) with regularization parameter chosen by 10-fold cross-validation following the ‘one standard error’ rule (Friedman, J., Hastie, T. & Tibshirani, R. Regularization Paths for Generalized Linear Models via Coordinate Descent. J Stat Softw. 33, 1-22 (2010); Hastie, T., Tibshirani, R. & Friedman, J. The Elements of Statistical Learning. Second cdn, (Springer, 2009)), selecting candidates among the identified CpGs. The associations of the score with all-cause, CVD, and cancer mortality were assessed first in the validation subset of the ESTHER cohort and then in the independent KORA cohort using multiple Cox regression models, adjusted for the covariates listed above (FIG. 1). All analyses were then repeated in men and women separately. In addition, to compare the predictive value of score with that of recently established methylomic predictors of ‘epigenetic age acceleration’ (i.e. Δage=DNAm age−chronological age), we assessed the associations of both score and Δage with all-cause mortality simultaneously. DNAm age was calculated according to two commonly applied algorithms introduced by Hannum et al. and Horvath et al. (cf. above).

The proportional hazards assumption was assessed by martingale-based residuals (Lin, D., Wei, L. & Ying, Z. Checking the Cox model with cumulative sums of martingale-based residuals. Biometrika. 80, 557-572 (1993)). No violations were detected. The LASSO regression analyses were conducted using the R-package ‘glmnet’. All other statistical analyses in the ESTHER study were carried out in SAS 9.4 (SAS Institute, Cary, N.C.) and the analyses in the KORA study were conducted in R (version 3.2.3).

### EXAMPLE 2

### Results

Study Population

Table 2 presents the baseline characteristics of the ESTHER study population. Of the 406 deaths in the case-cohort sample of the discovery panel, 90 were also included in the subcohort owing to random selection of subcohort at baseline. The time between blood sample collection and death ranged from 0.2 to 12.3 years [median (interquartile range (IQR), 7.4 (4.5-9.6) years] for these 406 participants. The corresponding figures for the 231 deaths in the validation panel were 0.2-13.8 years (range) and 8.6 (5.6-11.6) years [median (IQR)]. The characteristics of the participants in the subcohort of the discovery panel are similar as those of the participants in the validation panel, except that the proportion of women was larger in the subcohort than in the validation cohort. In comparison to those two subgroups, the group of deceased participants in the discovery panel featured higher proportions of men, smokers, old (>70 years) and inactive participants, and participants with prevalence of hypertension, diabetes, CVD, and cancer at baseline. The characteristics of the KORA study population are presented in Table 3. The average age was similar in KORA and ESTHER participants (61 vs. 62 years), but KORA participants had a much broader age range (31-82 years) than ESTHER participants (50-75 years).

Discovery and Validation of Mortality-Related CpGs

In the discovery phase, a total of 11,063 CpGs passed the genome-wide significance threshold (False Discovery Rate (FDR) <0.05) (FIG. 1). Associations with all-cause mortality were successfully replicated for 58 CpGs even after comprehensive confounder adjustment in the validation phase. Table 4 shows the results for the 58 CpGs. Methylation at the vast majority (49 of 58 CpGs) was inversely associated with mortality, with hazard ratios (HR) and 95% confidence intervals (95% CI) for a decrease in methylation by one standard deviation (SD) ranging from 1.16 (1.04-1.28) to 1.95 (1.29-2.94). HRs (95% CI) for the other 9 CpGs showing positive associations with mortality ranged from 0.60 (0.47-0.77) to 0.83 (0.71-0.97) per SD decrease in methylation. The 58 loci are located at 38 genes and 14 intergenic regions across 19 chromosomes. In addition to 3 CpGs within AHRR, 10 clusters within the identified sites were observed (Table 4), i.e., 1p21.2 (2 CpGs), 2q37 (2 CpGs), 3q11/12 (2 CpGs), 6p21 (4 CpGs), 11p15 (3 CpGs), 11q13 (3 CpGs), 17q21 (2 CpGs), 17q25 (2 CpGs), 19p13 (3 CpGs), and 19q13 (7 CpGs). A literature search in PubMed for genes containing the identified CpGs found evidence that these genes or their methylation are involved in a variety of major diseases, including diabetes (e.g., SARS, SQLE, NFE2L3, KCNQ1OT1, SOCS3), CVD (e.g. SARS, VCAM1, PLCL2, UTS2D, AHRR, 6p21.33, SQLE, KCNQ1OT1, SEMA7A, F2RL3, BCL3, PPP1R15A, PDE9A, MIR19A), various forms of cancers (e.g., SOCS3, SLC1A5, MIR19A, MIR10A, CALR, ERCC1, BCL3, SQLE, RARA, LAPTM5, INPP5A, CSGALNACT1, KCNQ1OT1, CDC42BPB, PDE9A, MKL1), neuropsychiatric disorders (FOSL2, ATL3, SHANK2, PPP1R15A) and HIV infection (e.g., GPR15, MIR10A) (Table 4). Several of those genes, such as SQLE, KCNQ1OT1, and SOCS3, have been suggested to play roles in multiple types of diseases.

Associations of Risk Factors with Mortality-Related CpGs

In the analyses of associations between the 58 CpGs and the covariates, differences in methylation levels with respect to age and sex were observed for 23 and 25 CpGs, respectively (Table 5). However, none of the 58 CpGs overlapped with previously identified aging-related sites. Forty-eight of the 58 CpGs were differentially methylated according to smoking exposure, and 22 of the 48 CpGs had also been found to be associated with smoking by previous EWASs (CpGs displayed in bold in Table 4). Five of the 48 smoking-associated CpGs and cg24397007 in FOSL2 were also associated with alcohol consumption (Table 5). Four of the 48 smoking-associated CpGs and cg08362785 in MKL1 were also associated with prevalent diabetes; of these 5 sites, cg18181703 in SOCS3 was also recently identified to be associated with type 2 diabetes (T2D) and cg23190089 is located at SLC22A18AS, a locus near to known methylation-regulated genes implicated in T2D (Travers, M. E. et al. Insights into the molecular mechanism for type 2 diabetes susceptibility at the KCNQ1 locus from temporal changes in imprinting status in human islets. Diabetes. 62, 987-992 (2013); Nilsson, E. et al. Altered DNA methylation and differential expression of genes influencing metabolism and inflammation in adipose tissue from subjects with type 2 diabetes. Diabetes. 63, 2962-2976 (2014)). In addition, 4 of the 48 smoking-associated CpGs, including 2 diabetes-associated sites (cg18181703 in SOCS3 and cg26470501 in BLC3), were also associated with prevalent cancer.

Mortality Risk Score and Validation

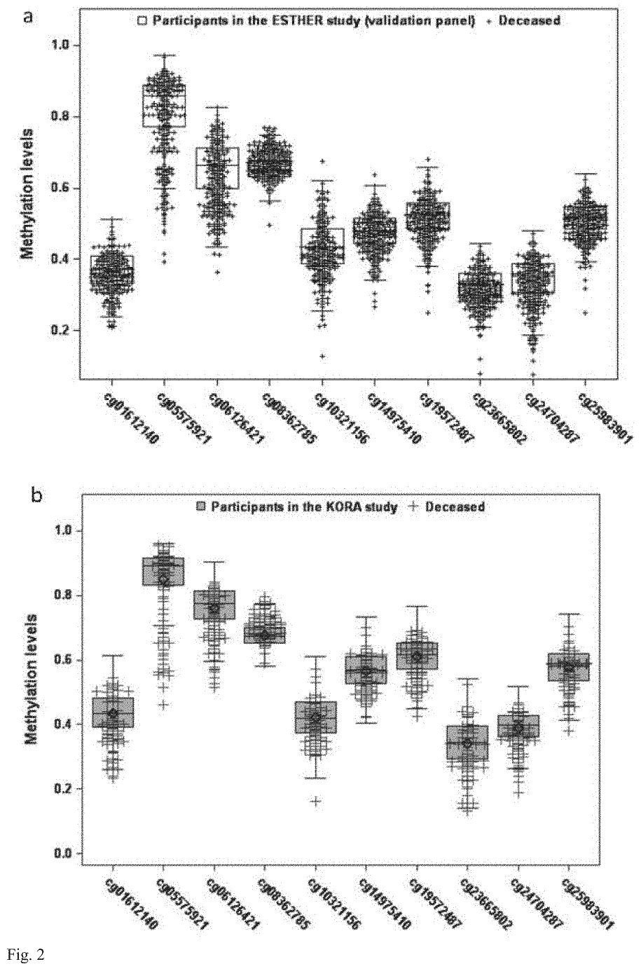

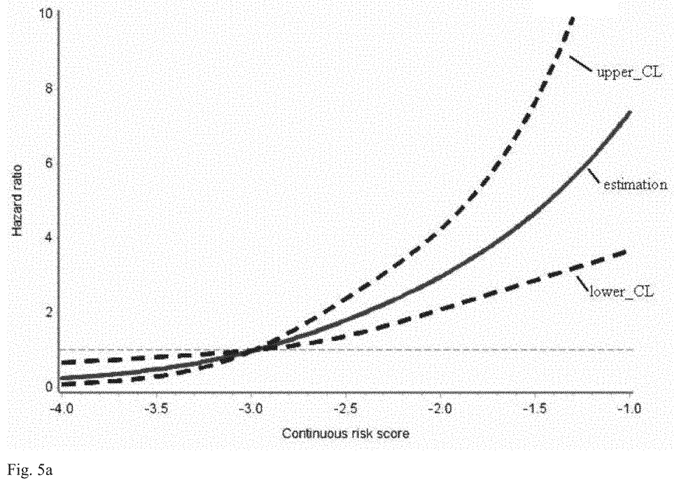

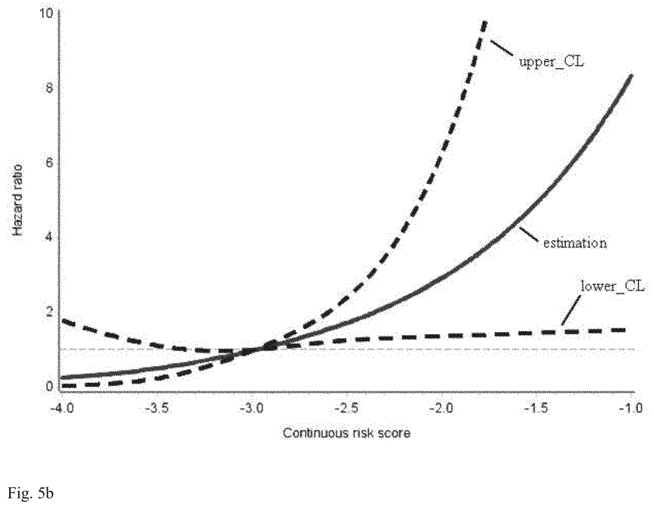

Ten CpGs (cg01612140, cg05575921, cg06126421, cg08362785, cg10321156, cg14975410, cg19572487, cg23665802, cg24704287, and cg25983901) were selected by LASSO regression. Preliminary analyses in ESTHER samples showed that ≥40% deaths occurred amongst participants with methylation levels in the highest quartile of cg08362785 (hypermethylated among deaths) or in the first quartile of the other 9 CpGs (demethylated among deaths) (FIG. 2a). We therefore used the 4^{th} quartile value of cg08362785 and 1^{st} quartile values of other 9 CpGs as the cutoff points to define aberrant methylation for each CpG (the exact cutoff points are listed in Table 6). Participants with aberrant methylation at 1 to 10 CpGs had a mortality score of 1 to 10, respectively, and participants without aberrant methylation at any of the 10 CpGs had score of 0. Table 7 shows the associations of score with all-cause mortality. Compared to participants with a score of 0, those who had a score of 1, 2-5, and 5+ had 2-, 3-, and 7-fold risk of dying, controlling for all the potential confounding factors. Analyses restricted to only older participants (≥60 years) yielded essentially the same risk estimates, e.g. HRs (95% CI) were 2.14 (1.02-4.47), 3.38 (1.68-6.80), and 7.44 (3.50-15.84), respectively, for a score of 1, 2-5, and 5+ vs. score=0. Similar patterns of distribution of deceased were also observed in KORA participants (FIG. 2b). Using the cut-off points from the ESTHER cohort defining aberrant methylation of 10 CpGs (Table 6), replicated analyses in the KORA cohort showed consistent patterns and similar risk estimates (Table 7). Crude HRs (95% CI) for participants with score of 1, 2-5, and 5+were 1.21 (0.37-3.97), 6.42 (2.55-16.18), and 19.29 (5.58-66.63), respectively, compared to score=0. In the fully adjusted model, 3- and 6-fold increases in mortality persisted for score levels of 2-5 and 5+, respectively. Using cut-off points (quartiles) of KORA itself defining aberrant methylation of the 10 CpGs to build the mortality score, risk estimates were larger than these derived from using ESTHER's cut-off points. For example, the HR (95% CI) in the fully adjusted model was 7.41 (1.61-34.07) for participants with score of 5+. In addition, a continuous risk score was computed through linear combination of LASSO regression coefficient weighted methylation values of the 10 CpGs (the combination formula is presented in FIG. 1). A similar trend that mortality monotonously increased with increasing continuous risk score was observed in both the ESTHER [risk score ranged from −3.92 to −0.72; median (IQR), −2.70 (−2.98-−2.35)] and the KORA cohorts [risk score ranged from −4.40 to −1.51; median (IQR), −3.15 (−3.41-−2.86)]. FIG. 5 shows the corresponding dose-response relationships derived from restricted cubic spline regression with adjustment for all the covariates again.

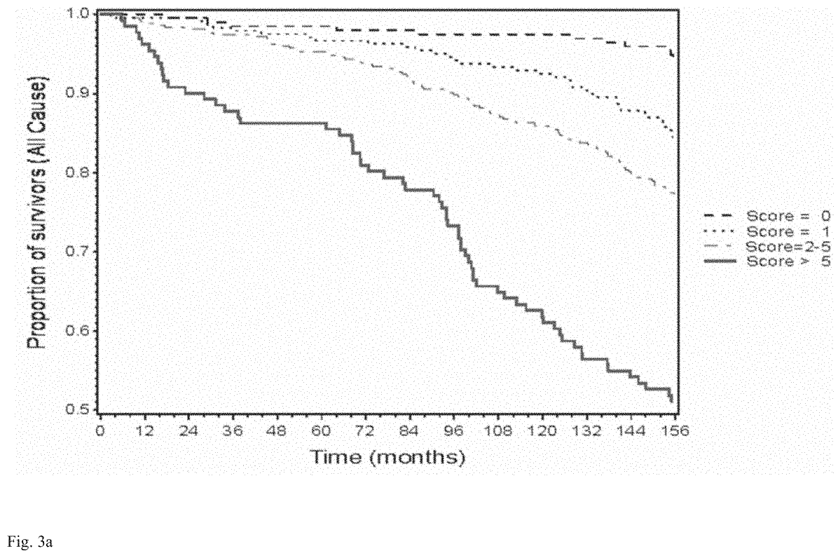

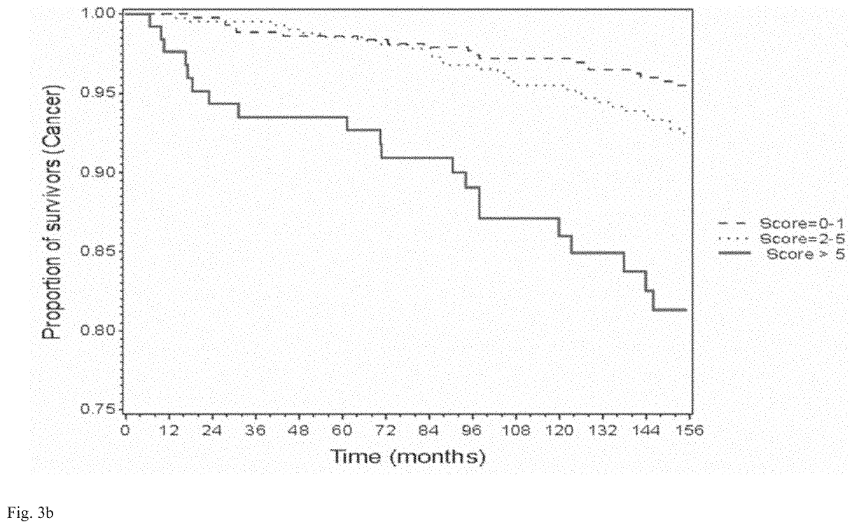

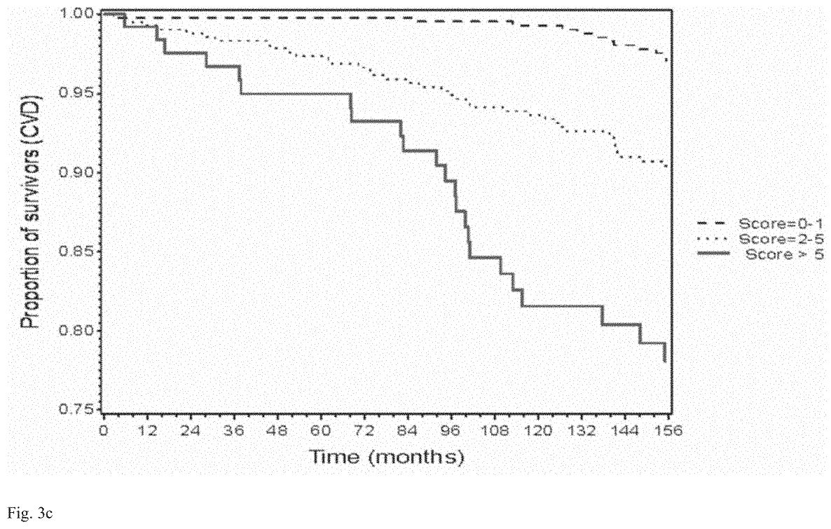

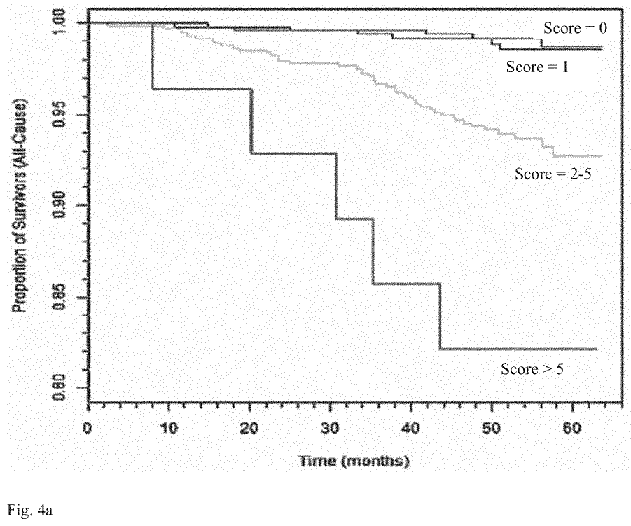

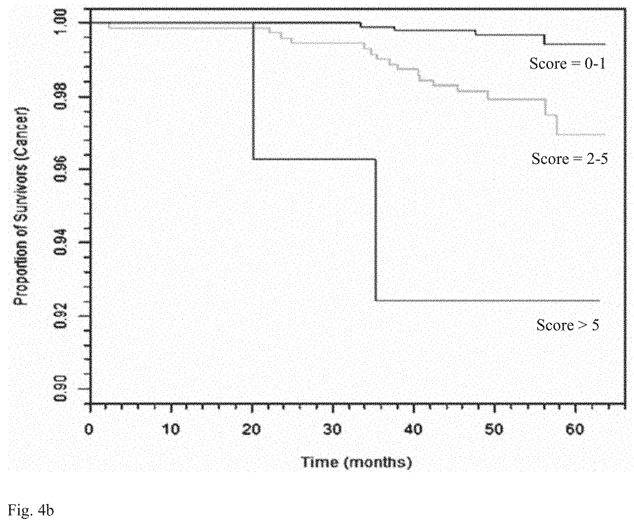

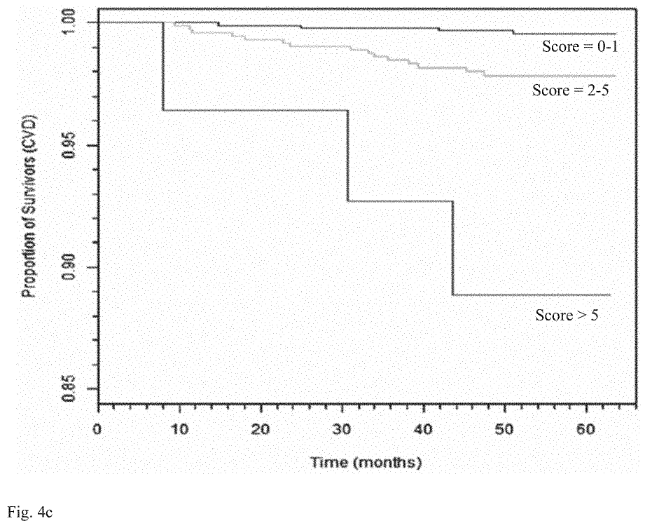

Sex-specific analyses indicated the associations with all-cause mortality to be stronger among women than among men in both cohorts (Table 8). Table 9 shows that the associations of score with CVD mortality were stronger than with cancer mortality in both cohorts. The corresponding survival curves in the ESTHER cohort are presented in FIG. 3. Similar survival curves were also obtained in the KORA cohort (FIG. 4).

Table 10 presents the associations of score with all-cause and cause-specific mortality in the ESTHER cohort under consideration of the epigenetic age acceleration (determined by Hannum et al.'s algorithm). The risk estimates of score for all three mortality outcomes were only very slightly attenuated by adjustment for the epigenetic age acceleration. On the contrary, HRs (95% CI) per 5-years of age acceleration dropped from 1.27 (1.10-1.46), 1.25 (0.98-1.59), and 1.34 (1.05-1.71), respectively, for all-cause, cancer and CVD mortality in the age- and sex-adjusted model to 1.08 (0.92-1.27), 1.15 (0.88-1.51), and 1.12 (0.85-1.48) in the full model. Similar results for the epigenetic age acceleration determined by Horvath et al.'s algorithm are presented in Table 11.

### EXAMPLE 3

### Discussion

In this EWAS and subsequent validation based on approximately 1,900 older adults with up to 14 years of follow-up, we identified blood DNAm of 58 CpGs across 19 chromosomes to be associated with all-cause mortality. While there is evidence that genes containing the identified CpGs are related to various types of common diseases, our study was the first to link DNAm of the vast majority of these genes to mortality in the general population. We additionally demonstrated that a risk score based on DNAm of 10 identified CpGs was a very strong predictor for all-cause, CVD, and cancer mortality, and we confirmed this finding in an independent cohort study. None of the newly identified CpGs overlapped with previously established aging-related CpGs, and the strong associations of score with mortality were also independent from the epigenetic clock.

Of the 58 identified CpGs, the top one locus showing the most significant association with mortality was cg05575921 in AHRR, followed by cg21161138 in AHRR, cg26963277 in KCNQ1OT1, cg19859270 in GPR15, and cg03636183 in F2RL3, cg19572487 in RARA, and cg06126421 in 6p21.33. All these CpGs (except cg26963277 in KCNQ1OT1) were also the top signals in previous EWASs on smoking. In addition to the 22 CpGs identified to be associated with smoking in previous EWASs, another 26 of the 58 CpGs were also smoking-associated in the current study. Furthermore, even though a few other CpGs were found to be associated with alcohol consumption, diabetes or cancer, such as cg18181703 in SOCS3 and cg26470501 in BCL3, most of them also showed associations with smoking exposure in our analyses. These findings suggest that tobacco smoking is the strongest factor leaving imprints on DNAm such that smoking rather than other common health risk factors accounts for the major burden of morbidity and mortality involving epigenetic programming. Regardless of the underlying mechanisms which remain to be elucidated in further research, it appears worthwhile pointing out that prevention of or intervention on smoking-related DNAm changes may provide major improvement in premature death prevention, given the reversibility of smoking-induced methylomic aberrations (Zhang, Y., Yang, R., Burwinkel, B., Breitling, L. P. & Brenner, H. F2RL3 Methylation as a Biomarker of Current and Lifetime Smoking Exposures. Environ Health Perspect. 122, 131-137 (2014); Guida, F. et al. Dynamics of smoking-induced genome-wide methylation changes with time since smoking cessation. Hum Mol Genet. 24, 2349-2359 (2015)).

Compared to genetic variants related to longevity identified by GWAS, which typically show very small effect sizes of single SNPs, in particular in general population samples, the effect size of even single CpGs identified in the current EWAS were substantial, with HRs≥1.17 or ≤0.83 per SD increase of methylation, resulting in the strong overall prediction when combining these CpGs in a risk score. To our knowledge, no comparably strong prediction of mortality based on genetic data has been identified, suggesting that epigenetic data might be more informative for mortality prediction than genetic data.

The recently established epigenetic clock (DNAm age) has received growing attention as an increasing number of studies have uncovered it to be a proxy of biological aging and thus potentially providing a measure for assessing health and mortality. Intriguingly, we targeted mortality-related DNAm changes and did not find any overlap with previously established CpGs that are used to determine the DNAm age. Our findings are in line with evidence suggesting that DNAm involved in aging or health-related outcomes are mostly regulated by DNAm regions other than the established age-related DNAm.

In the analysis we did not exclude probes that might be affected by known SNPs as annotated by ‘Infinium HD Methylation SNP List’ (support.illumina.com/array/array_kits/infinium_humanmethylation450_beadchip_kit/downloads.html). We later retrieved data of 32 such SNPs for 24 identified CpGs in 581 ESTHER participants of the validation set. Only one SNP-CpG pair (rs524-cg03707168) showed a significant association. However, no association was observed between rs524 and all-cause mortality irrespective of controlling for DNAm of cg03707168, whereas the strong association of cg03707168 with mortality did not change when controlling for rs524. And no interaction was detected between rs524 and cg03707168 in relation to mortality.

TABLE 2
Characteristics of study population at baseline

| | Discovery panel | | |
|---|---|---|---|
| | N (%) | | Validation panel |
| | All deaths | Subcohort | N (%) |
| Characteristics | (n = 406) | (n = 548)^{a} | Cohort (n = 1000) |
| Sex | | | |
| Male | 224 (55.2) | 212 (38.7) | 500 (50.0) |
| Female | 182 (44.8) | 336 (61.3) | 500 (50.0) |
| Age (years) | | | |
| 50-60 | 84 (20.7) | 179 (32.7) | 339 (33.9) |
| 60-64 | 97 (23.9) | 159 (29.0) | 289 (28.9) |
| 65-69 | 113 (27.8) | 127 (23.2) | 226 (22.6) |
| 70-75 | 112 (27.6) | 83 (15.1) | 146 (14.6) |
| Smoking status^{b} | | | |
| Never smoker | 155 (39.6) | 251 (47.3) | 469 (48.0) |
| Former smoker | 136 (34.8) | 180 (33.9) | 323 (33.0) |
| Current smoker | 100 (25.6) | 100 (18.8) | 186 (19.0) |
| Body mass index (kg/m²)^{c} | | | |
| Underweight (<18.5) | 5 (1.2) | 1 (0.2) | 8 (0.8) |
| Normal weight (18.5-<25.0) | 117 (28.9) | 166 (30.3) | 243 (24.4) |
| Overweight (25.0-<30.0) | 173 (42.7) | 235 (42.9) | 483 (48.5) |
| Obesity (≥30.0) | 110 (27.2) | 146 (26.6) | 263 (26.4) |
| Physical activity^{d} | | | |
| Inactive | 108 (26.7) | 114 (20.9) | 203 (20.3) |
| Low | 205 (50.6) | 268 (49.1) | 438 (43.8) |
| Medium/high | 92 (22.7) | 164 (30.0) | 358 (35.8) |
| Prevalence of major diseases | | | |
| Hypertension | 278 (68.5) | 308 (56.2) | 589 (58.9) |
| Diabetes^{e} | 108 (26.6) | 95 (17.4) | 162 (16.2) |
| Cardiovascular disease^{e} | 120 (29.6) | 97 (17.7) | 182 (18.2) |
| Cancer | 57 (14.0) | 27 (4.9) | 66 (6.6) |

^{a}The subcohort included 90 deaths due to random selection at baseline irrespective of death status during follow-up;
^{b}Data missing for 27 and 22 subjects, respectively, in discovery and validation panels.
^{c}Data missing for 1 and 3 subjects, respectively, in discovery and validation panels.
^{d}Data missing for 3 and 1 subjects, respectively, in discovery and validation panels.
^{e}Data missing for 1 subject in both discovery and validation panels.

TABLE 3
Characteristics of the KORA study population at baseline

| Characteristic | N (%)/Mean (Standard deviation) |
|---|---|
| Male (%) | 845 (48.9) |
| Age (years) | 61.0 (8.9) |
| Never/former/current smokers (%)^{a} | 721 (41.8)/754 (43.7)/250 (14.5) |
| Body mass index (kg/m²) | 28.1 (4.8) |
| Physically active (%)^{a} | 985 (57.1) |
| Hypertension (%)^{b} | 789 (45.8) |
| Diabetes (%)^{c} | 161 (9.3) |
| Coronary heart disease (%)^{c} | 105 (6.0) |
| Cancer (%)^{c} | 154 (8.9) |
| Total cholesterol (mg/dl) | 221.9 (39.2) |
| Systolic blood pressure | 124.8 (18.7) |

^{a}Data missing for 2 subjects;
^{b}Data missing for 4 subjects;
^{c}Data missing for 1 subject.

TABLE 4
Association of 58 CpGs with all-cause mortality in the validation panel

| CpG site | HR (95% CI)^{a} | FDR | Chr position (GRCh37/hg19) | Gene name | Gene related major diseases^{b} |
|---|---|---|---|---|---|
| cg03725309 | 1.34 (1.10-1.62) | 0.0450 | 1p13.3 (chr1: 109757585) | SARS | T2D (M); coronary artery disease |
| cg25763716 | 1.29 (1.02-1.63) | 0.0486 | 1p21.2 (chr1: 101184304) | VCAM1 | atherosclerosis; MI; tumor invasion |
| cg13854219 | 1.51 (1.05-2.17) | 0.0399 | 1p21.2 (chr1: 101757037) | | |
| cg25189904 | 1.18 (1.02-1.38) | 0.0450 | 1p31.3 (chr1: 68299493) | GNG12 | endometrial cancer |
| cg15459165 | 0.60 (0.47-0.77) | 0.0035 | 1p35.2 (chr1: 31223850) | LAPTM5 | lung cancer (M); NB (M); multiple myeloma (M) |
| cg19266329 | 1.33 (1.14-1.55) | 0.0179 | 1q21.1 (chr1: 145456128) | | |
| cg24397007 | 1.28 (1.08-1.53) | 0.0483 | 2p23.2 (chr2: 28619095) | FOSL2 | Parkinson's disease (M); breast cancer |
| cg23079012 | 1.16 (1.04-1.28) | 0.0008 | 2p25.1 (chr2: 8343711) | | |
| cg27241845 | 1.23 (1.06-1.44) | 0.0222 | 2q37.1 (chr2: 233250371) | | |
| cg06905155 | 1.20 (1.05-1.36) | 0.0450 | 2q37.3 (chr2: 240723946) | | |
| cg16503724 | 0.77 (0.64-0.94) | 0.0484 | 3p24.3 (chr3: 17130667) | PLCL2 | renal cell carcinoma (M); MI; systemic sclerosis |
| cg19859270 | 1.32 (1.13-1.54) | 0.0001 | 3q11.2 (chr3: 98251295) | GPR15 | HIV |
| cg02657160 | 1.22 (1.07-1.38) | 0.0084 | 3q12.1 (chr3: 98311063) | CPOX | |
| cg14975410 | 1.20 (1.04-1.38) | 0.0372 | 3q26.31 (chr3: 171180070) | | |
| cg14855367 | 1.23 (1.08-1.40) | 0.0463 | 3q28 (chr3: 191048309) | UTS2D | coronary artery disease |
| cg05575921 | 1.51 (1.25-1.84) | 4.25E−07 | 5p15.33 (chr5: 373378) | AHRR | lung cancer (M); atherosclerosis (M) |
| cg14817490 | 1.19 (1.01-1.42) | 0.0260 | 5p15.33 (chr5: 392920) | AHRR | CVD/cancer death (M) |
| cg21161138 | 1.23 (1.05-1.44) | 3.07E−05 | 5p15.33 (chr5: 399361) | AHRR | |
| cg12513616 | 1.17 (1.01-1.36) | 0.0280 | 5q35.3 (chr5: 177370977) | | |
| cg20732076 | 1.25 (1.05-1.50) | 0.0217 | 6p21.1 (chr6: 42335232) | TRERF1 | breast cancer |
| cg25285720 | 1.25 (1.06-1.46) | 0.0488 | 6p21.32 (chr6: 32919434) | HLA-DMA | ovarian cancer (M) |
| cg06126421 | 1.33 (1.10-1.60) | 0.0008 | 6p21.33 (chr6: 30720081) | | lung cancer (M); CVD/cancer death (M) |
| cg15342087 | 1.17 (1.01-1.36) | 0.0450 | 6p21.33 (chr6: 30720210) | | |
| cg01612140 | 1.40 (1.14-1.72) | 0.0244 | 6q14.1 (chr6: 78166437) | | |
| cg25983901 | 1.19 (1.02-1.40) | 0.0450 | 7p12.3 (chr7: 46972700) | | |
| cg12510708 | 1.33 (1.06-1.67) | 0.0241 | 7p15.2 (chr7: 26193806) | NFE2L3 | T2D (M); breast cancer (M) |
| cg26286961 | 1.27 (1.10-1.47) | 0.0260 | 8p21.3 (chr8: 19460209) | CSGALNACT1 | FV-PTC; multiple myeloma |
| cg00285394 | 1.20 (1.05-1.36) | 0.0217 | 8q24.13 (chr8: 126011954) | SQLE | T2D/CVD (M); breast cancer (M); lung/prostate cancer |
| cg01140244 | 0.69 (0.54-0.89) | 0.0450 | 10q26.3 (chr10: 134498960) | INPP5A | brain tumor; Cutaneous squamous cell carcinoma |
| cg23190089 | 1.40 (1.08-1.82) | 0.0450 | 11p15.4 (chr11: 2920209) | SLC22A18AS | breast cancer (M) |
| cg07123182 | 1.26 (1.11-1.44) | 0.0003 | 11p15.5 (chr11: 2722391) | KCNQ1OT1 | T2D (M); CRC (M); MI; breast cancer |
| cg26963277 | 1.31 (1.14-1.49) | 3.07E−05 | 11p15.5 (chr11: 2722408) | KCNQ1OT1 | |
| cg18550212 | 1.57 (1.22-2.01) | 0.0217 | 11q13.1 (chr11: 63435428) | ATL3 | neuropathy |
| cg10321156 | 1.20 (1.02-1.42) | 0.0450 | 11q13.1 (chr11: 63687223) | | |
| cg25193885 | 0.78 (0.65-0.93) | 0.0100 | 11q13.3 (chr11: 70328867) | SHANK2 | prostate cancer (M); neuropsychiatric disorders |
| cg07986378 | 1.27 (1.03-1.57) | 0.0483 | 12p13.2 (chr12: 11898285) | ETV6 | hematopoiesis and malignant transformation |
| cg23665802 | 1.39 (1.13-1.71) | 0.0122 | 13q31.3 (chr13: 92002338) | MIR19A | CVD; lung/gastric/breast/bladder/cervical cancer/ |
| | | | | | CRC/HCC |
| cg04987734 | 0.81 (0.70-0.94) | 0.0266 | 14q32.32 (chr14: 103415874) | CDC42BPB | tumor cell invasion, e.g. CRC (M); breast cancer |
| cg19459791 | 0.83 (0.71-0.97) | 0.0483 | 15q22.31 (chr15: 65363023) | | |
| cg00310412 | 1.26 (1.07-1.47) | 0.0241 | 15q24.1 (chr15: 74724919) | SEMA7A | multiple sclerosis; lung/liver fibrosis |
| cg26709988 | 1.95 (1.29-2.94) | 0.0092 | 16q24.1 (chr16: 84860919) | CRISPLD2 | |
| cg23842572 | 0.75 (0.62-0.91) | 0.0194 | 17p11.2 (chr17: 17030253) | MPRIP | cancer cell invasion |
| cg19572487 | 1.26 (1.07-1.49) | 0.0003 | 17q21.2 (chr17: 38476025) | RARA | breast cancer (M); hepatocellular/ |
| | | | | | thyroid carcinomas (M) |
| cg01572694 | 1.36 (1.12-1.67) | 0.0311 | 17q21.32 (chr17: 46657555) | MIR10A | lung/gastric/breast/colon/pancreatic/brain |
| | | | | | cancer/HCC; HIV |
| cg08546016 | 1.39 (1.13-1.70) | 0.0372 | 17q25.1 (chr17: 72776239) | TMEM104 | |
| cg18181703 | 1.24 (1.07-1.44) | 0.0214 | 17q25.3 (chr17: 76354622) | SOCS3 | T2D (M); lung/pancreatic/cervical/endometrial/prostate |
| | | | | | cancer/HNSCC/HCC/CRC/melanoma/ |
| | | | | | glioblastoma/leukemia (M) |
| cg03636183 | 1.27 (1.06-1.51) | 0.0003 | 19p13.11 (chr19: 17000586) | F2RL3 | lung cancer (M); CVD/cancer death(M) |
| cg24704287 | 1.31 (1.06-1.61) | 0.0329 | 19p13.13 (chr19: 13951482) | | |
| cg11341610 | 1.29 (1.04-1.59) | 0.0421 | 19p13.2 (chr19: 13050932) | CALR | Lung/gastric/pancreatic/prostate/ovarian cancers/NB |
| cg14085840 | 1.45 (1.10-1.91) | 0.0486 | 19q13.2 (chr19: 40939429) | | |
| cg26470501 | 1.20 (1.04-1.39) | 0.0351 | 19q13.32 (chr19: 45252955) | BCL3 | CVD; lung/breast/prostate cancer/CRC |
| cg05492306 | 1.39 (1.07-1.80) | 0.0217 | 19q13.32 (chr19: 45927594) | ERCC1 | lung/breast cancer(M); HNSCC/gastric cancer |
| cg25607249 | 1.41 (1.10-1.81) | 0.0345 | 19q13.32 (chr19: 47288040) | SLC1A5 | T2D (M); Lung/pancreatic/breast/prostate |
| | | | | | cancer/CRC/NB/ |
| cg01406381 | 1.52 (1.19-1.95) | 0.0054 | 19q13.32 (chr19: 47288263) | SLC1A5 | melanoma/renal cell carcinoma |
| cg07626482 | 1.19 (1.03-1.38) | 0.0463 | 19q13.32 (chr19: 47289503) | SLC1A5 | |
| cg03707168 | 1.29 (1.02-1.63) | 0.0311 | 19q13.33 (chr19: 49379127) | PPP1R15A | neurological diseases; myocardial ischaemia |
| cg25491402 | 0.65 (0.47-0.90) | 0.0496 | 21q22.3 (chr21: 44101491) | PDE9A | lung cancer (M); CVD; breast cancer |
| cg08362785 | 0.63 (0.51-0.78) | 0.0003 | 22q13.1 (chr22: 40814879) | MKL1 | lung/breast cancer(M); lung/liver fibrosis(M) |

Abbreviations Table 4:
CI, confidence interval;
CVD, cardiovascular disease;
FDR, false discovery rate;
FV-PTC, follicular variant of papillary thyroid carcinoma;
HCC, hepatocellular carcinoma;
HIV, human immunodeficiency virus;
HNSCC, head and neck squamous cell carcinoma;
HR, hazard ratio;
MI, myocardial infarction;
NB, neuroblastoma; T2D, type 2 diabetes;
Bold printed CpGs (n = 22) are sites identified to be associated with smoking in both the current and previous epigenome-wide association studies.
Underscored CpGs (n = 10) were selected to develop the mortality risk score.
Bold printed ‘Chr position’ indicates clusters of identified CpGs.
^{a}Hazard ratios for a decrease in methylation by one standard deviation; model adjusted for age, sex, smoking status, BMI, physical activity, systolic blood pressure, total cholesterol, hypertension, and prevalent cardiovascular disease, diabetes, and cancer at baseline;
^{b}M refers to diseases which have been reported to be related to methylation of the gene; detailed descriptions of gene function and relevant diseases are listed in Supplementary Table 1.

TABLE 5
List of CpGs associated with lifestyle factors and prevalent diseases at baseline

| | | FDR^{a} | | | | | |
|---|---|---|---|---|---|---|---|
| | | sex | age | smoking status | alcohol consumption | diabetes | Cancer |
| CpG site | Gene name | (n = 23 CpGs) | (n = 23 CpGs) | (n = 48 CpGs) | (n = 6 CpGs) | (n = 5 CpGs) | (n = 5 CpGs) |
| cg03725309 | SARS | | 4.25E−08 | 0.0138 | 2.24E−05 | | |
| cg25763716 | VCAM1 | | 0.0022 | | | | |
| cg13854219 | | | | 0.0003 | | | |
| cg25189904 | GNG12 | 2.19E−08 | | 4.47E−25 | | | |
| cg15459165 | LAPTM5 | | | 0.0007 | | | |
| cg19266329 | | 0.0133 | | 5.38E−05 | 0.0008 | 0.0014 | |
| cg24397007 | FOSL2 | | | | 0.0002 | | |
| cg23079012 | | 9.99E−05 | | 9.59E−24 | | | |
| cg27241845 | | 2.67E−27 | | 6.50E−23 | | | |
| cg06905155 | | | | | | | 0.0080 |
| cg16503724 | PLCL2 | 0.0232 | | 3.95E−12 | | | |
| cg19859270 | GPR15 | 0.0029 | | 1.35E−51 | | | |
| cg02657160 | CPOX | | | 1.75E−16 | | | |
| cg14975410 | | | 0.0021 | 0.0033 | | | |
| cg05575921 | AHRR | 2.69E−10 | | 2.18E−135 | | | |
| cg14817490 | AHRR | | 0.0370 | 6.35E−52 | | | |
| cg21161138 | AHRR | 0.0063 | | 3.01E−69 | | | |
| cg12513616 | | 2.19E−08 | | 9.42E−18 | | | |
| cg20732076 | TRERF1 | | 0.0283 | 3.52E−09 | 0.0230 | | |
| cg06126421 | | 5.92E−08 | 0.0198 | 1.79E−61 | | | |
| cg15342087 | | 0.0028 | | 3.84E−40 | | | |
| cg01612140 | | 6.95E−08 | | 0.0103 | | | |
| cg25983901 | | | 0.0194 | 0.0015 | 0.0002 | | |
| cg12510708 | NFE2L3 | | 0.0021 | 0.0093 | | | |
| cg00285394 | SQLE | 0.0017 | | 0.0021 | | | |
| cg01140244 | INPP5A | | 0.0022 | | | | |
| cg23190089 | SLC22A18AS | 0.0014 | 1.74E−05 | 2.14E−09 | | 0.0199 | |
| cg07123182 | KCNQ1OT1 | | 0.0198 | 2.58E−17 | | | |
| cg26963277 | KCNQ1OT1 | 0.0104 | | 1.51E−21 | | | |
| cg18550212 | ATL3 | | 0.0021 | 0.0277 | | | |
| cg10321156 | | 6.93E−06 | | 0.0173 | | | |
| cg25193885 | SHANK2 | 0.0422 | | 0.0372 | | | |
| cg07986378 | ETV6 | 0.0488 | | 7.51E−20 | | | |
| cg23665802 | MIR19A | | 0.0004 | 3.74E−05 | | | |
| cg04987734 | CDC42BPB | 0.0169 | | 0.0050 | | | |
| cg19459791 | | | | 2.32E−05 | | | |
| cg00310412 | SEMA7A | 0.0002 | | 1.62E−20 | | | |
| cg23842572 | MPRIP | | 0.0370 | 5.85E−06 | | | |
| cg19572487 | RARA | 4.68E−10 | 0.0018 | 7.66E−36 | | | |
| cg01572694 | MIR10A | | | 0.0479 | | | |
| cg08546016 | TMEM104 | | | 0.0338 | | | |
| cg18181703 | SOCS3 | | | 0.0125 | | 0.0373 | 0.0303 |
| cg03636183 | F2RL3 | 0.00012 | | 1.01E−95 | | | 0.0145 |
| cg24704287 | | | 0.0030 | 3.85E−07 | | | 0.0117 |
| cg11341610 | CALR | 0.0187 | 0.0026 | 0.0021 | | | |
| cg14085840 | | 0.0492 | | 0.0003 | | | |
| cg26470501 | BCL3 | | 0.0038 | 6.77E−06 | | 0.0199 | 0.0117 |
| cg05492306 | ERCC1 | | 8.84E−05 | 2.27E−06 | | | |
| cg25607249 | SLC1A5 | | 0.0407 | 9.34E−05 | | | |
| cg01406381 | SLC1A5 | | 0.0070 | 5.85E−06 | | | |
| cg07626482 | SLC1A5 | | | 2.32E−05 | 5.53E−07 | | |
| cg03707168 | PPP1R15A | | 0.0030 | 2.70E−31 | | | |
| cg08362785 | MKL1 | 1.02E−05 | 2.05E−07 | | | 0.0445 | |

^{a}FDR, False discovery rate; FDR < 0.05 in mixed regression analysis with each validated CpG as dependent variable and age (years), sex, smoking status (never/former/current smokers), alcohol consumption, body mass index ((<25 kg/m2/25.0-<30.0 kg/m2/≥30.0 kg/m2), physical activity (inactive/low/medium or high), total cholesterol, prevalence of hypertension, cardiovascular disease, diabetes, and cancer as independent variables, controlling for batch effects and leukocyte composition.

TABLE 6
Cutoff points for defining aberrant methylation for the 10 CpG

| CpG sites | cutoff points |
|---|---|
| cg01612140 | ≤0.33962 |
| cg05575921 | ≤0.77332 |
| cg06126421 | ≤0.59937 |
| cg08362785 | ≥0.67841 |
| cg10321156 | ≤0.38737 |
| cg14975410 | ≤0.44420 |
| cg19572487 | ≤0.48629 |
| cg23665802 | ≤0.29927 |
| cg24704287 | ≤0.30730 |
| cg25983901 | ≤0.48522 |

TABLE 7
Association of the risk score with all-cause mortality in the ESTHER and KORA study

| | | | | | | HR (95% CI) | | | | | |
|---|---|---|---|---|---|---|---|---|---|---|---|
| Study | Mortality score^{a} | Nₜₒₜₐₗ | Cases | PY | IR^{b} | Model 1^{c} | | Model 2^{d} | | Model 3^{e} | |
| ESTHER study | 0 | 199 | 14 | 2690.69 | 0.52 | Ref. | | Ref. | | Ref. | |
| | 1 | 242 | 41 | 3144.50 | 1.30 | 2.55 | (1.39-4.68) | 2.04 | (1.11-3.75) | 2.16 | (1.10-4.24) |
| | 2-5 | 426 | 105 | 5300.86 | 1.98 | 3.93 | (2.25-6.86) | 3.18 | (1.81-5.59) | 3.42 | (1.81-6.46) |
| | >5 | 131 | 70 | 1348.99 | 5.19 | 10.89 | (6.13-19.35) | 7.64 | (4.21-13.85) | 7.36 | (3.69-14.68) |
| KORA study | 0 | 487 | 5 | 2163.01 | 0.23 | Ref. | | Ref. | | Ref. | |
| | 1 | 490 | 6 | 2147.91 | 0.28 | 1.21 | (0.37-3.97) | 0.93 | (0.28-3.05) | 0.71 | (0.20-2.46) |
| | 2-5 | 722 | 45 | 3070.1 | 1.47 | 6.42 | (2.55-16.18) | 3.95 | (1.53-10.19) | 3.19 | (1.22-8.35) |
| | >5 | 28 | 5 | 114.32 | 4.37 | 19.29 | (5.58-66.63) | 10.95 | (3.09-38.84) | 5.93 | (1.49-23.69) |

Abbreviations: HR, hazard ratio; CI, confidence interval; CVD, cardiovascular disease; IR, incidence rate; PY, person-years; Ref., reference category.
^{a}Score was based on methylation of 10 CpGs (cg01612140, cg05575921, cg06126421, cg08362785, cg10321156, cg14975410, cg19572487, cg23665802, cg24704287, cg25983901) using their respective first quartile values (cg08362785: using its highest quartile) among the ESTHER participants as the cutoff points to define aberrant methylation; Score 0-10 refer to simultaneously aberrant methylation at 0 to 10 CpGs;
^{b}Incidence rate per 100 person-years;
^{c}Model 1: without adjustment;
^{d}Model 2: adjusted for chronological age and sex;
^{e}Model 3: like model 2, additionally adjusted for smoking status, BMI, physical activity, alcohol consumption, systolic blood pressure, total cholesterol, hypertension, and prevalent cardiovascular disease, diabetes, and cancer at baseline.

TABLE 8
Sex-specific associations of risk score with all-cause mortality in the ESTHER study and KORA study

| | | | | | | HR (95% CI) | | | | | |
|---|---|---|---|---|---|---|---|---|---|---|---|
| Group | Mortality score^{a} | Nₜₒₜₐₗ | Cases | PY | IR^{b} | Model 1^{c} | | Model 2^{d} | | Model 3^{e} | |
| ESTHER-Men | 0 | 61 | 4 | 808.09 | 0.50 | Ref. | | Ref. | | Ref. | |
| | 1 | 97 | 23 | 1217.62 | 1.89 | | | | | | |
| | 2-5 | 236 | 62 | 2888.82 | 2.15 | 1.63 | (1.04-2.55) | 1.51 | (0.96-2.37) | 1.50 | (0.93-2.43) |
| | >5 | 105 | 54 | 1068.33 | 5.05 | 3.94 | (2.48-6.26) | 3.32 | (2.08-5.29) | 3.04 | (1.76-5.25) |
| ESTHER-Women | 0 | 138 | 10 | 1882.60 | 0.53 | Ref. | | Ref. | | Ref. | |
| | 1 | 145 | 18 | 1926.88 | 0.93 | | | | | | |
| | 2-5 | 189 | 43 | 2412.04 | 1.78 | 2.51 | (1.56-4.05) | 2.48 | (1.54-3.99) | 2.67 | (1.59-4.48) |
| | >5 | 26 | 16 | 280.65 | 5.70 | 9.17 | (4.95-16.98) | 8.36 | (4.50-15.51) | 8.31 | (3.76-18.35) |
| KORA-Men | 0 | 177 | 1 | 795.15 | 0.13 | Ref. | | Ref. | | Ref. | |
| | 1 | 220 | 3 | 958.78 | 0.31 | | | | | | |
| | 2-5 | 428 | 36 | 1810.53 | 1.99 | 8.86 | (3.15-24.9) | 7.16 | (2.53-20.3) | 6.17 | (2.13-17.84) |
| | >5 | 20 | 4 | 80.41 | 4.97 | 22.28 | (5.57-89.1) | 16.9 | (4.19-68.19) | 10.46 | (2.26-48.41) |
| KORA-Women | 0 | 310 | 4 | 1367.86 | 0.29 | Ref. | | Ref. | | Ref. | |
| | 1 | 270 | 3 | 1189.13 | 0.25 | | | | | | |
| | 2-5 | 294 | 9 | 1259.57 | 0.71 | 2.61 | (0.97-7.01) | 2.1 | (0.78-5.66) | 1.86 | (0.61-5.63) |
| | >5 | 8 | 1 | 33.91 | 2.95 | 10.85 | (1.33-88.21) | 17.73 | (2.08-151.49) | 14.34 | (1.02-201.11) |

Abbreviations: HR, hazard ratio; CI, confidence interval; CVD, cardiovascular disease; IR, incidence rate; PY, person-years; Ref, reference category.
^{a}Score was based on methylation of 10 CpGs (cg01612140, cg05575921, cg06126421, cg08362785, cg10321156, cg14975410, cg19572487, cg23665802, cg24704287, cg25983901) using their respective first quartile values (cg08362785: using its highest quartile) among the ESTHER participants as the cutoff points to define aberrant methylation; Score 0-10 refer to simultaneously aberrant methylation at 0 to 10 CpGs;
^{b}Incidence rate per 100 person-years;
^{c}Model 1: without adjustment;
^{d}Model 2: adjusted for chronological age.
^{f}Model 3: like model 2, additionally adjusted for smoking status, BMI, physical activity, alcohol consumption, systolic blood pressure, total cholesterol, hypertension, and prevalent cardiovascular disease, diabetes, and cancer at baseline.

TABLE 9
Associations of the risk score with cancer and CVD mortality in the ESTHER and KORA study

| | | | | | | | HR (95% CI) | | |
|---|---|---|---|---|---|---|---|---|---|
| Outcome | Study | Mortality score^{a} | Nₜₒₜₐₗ | Cases | PY | IR^{b} | Model 1^{c} | Model 2^{d} | Model 3^{e} |
| Cancer mortality | ESTHER | 0 | 199 | 8 | 2690.69 | 0.30 | Ref. | Ref. | Ref. |
| | | 1 | 242 | 17 | 3144.50 | 0.54 | | | |

| | | | | | | | | | | |
|---|---|---|---|---|---|---|---|---|---|---|
| 2-5 | 426 | 31 | 5300.86 | 0.58 | 1.38 | (0.82-2.34) | 1.24 | (0.72-2.11) | 1.21 | (0.68-2.15) |
| >5 | 131 | 22 | 1348.99 | 1.63 | 4.11 | (2.31-7.30) | 3.12 | (1.69-5.78) | 2.57 | (1.27-5.21) |

| | | | | | | | | |
|---|---|---|---|---|---|---|---|---|
| KORA | 0 | 487 | 3 | 2163.01 | 0.14 | Ref. | Ref. | Ref. |
| | 1 | 490 | 1 | 2147.91 | 0.05 | | | |

| | | | | | | | | | | |
|---|---|---|---|---|---|---|---|---|---|---|
| 2-5 | 722 | 16 | 3070.1 | 0.52 | 5.78 | (1.93-17.31) | 4.28 | (1.39-13.13) | 3.16 | (1.01-9.85) |
| >5 | 28 | 2 | 114.32 | 1.75 | 19.42 | (3.56-106.06) | 14.74 | (2.6-83.69) | 5.74 | (0.84-39.42) |

| | | | | | | | | | |
|---|---|---|---|---|---|---|---|---|---|
| CVD mortality | ESTHER | 0 | 199 | 4 | 2690.69 | 0.15 | Ref. | Ref. | Ref. |
| | | 1 | 242 | 9 | 3144.50 | 0.29 | | | |

| | | | | | | | | | | |
|---|---|---|---|---|---|---|---|---|---|---|
| 2-5 | 426 | 43 | 5300.86 | 0.81 | 3.69 | (1.99-6.87) | 3.41 | (1.82-6.40) | 4.00 | (1.96-8.15) |
| >5 | 131 | 25 | 1348.99 | 1.85 | 9.04 | (4.62-17.70) | 7.19 | (3.54-14.62) | 9.12 | (3.89-21.39) |

| | | | | | | | | |
|---|---|---|---|---|---|---|---|---|
| KORA | 0 | 487 | 2 | 2163.01 | 0.09 | Ref. | Ref. | Ref. |

| 1 | 490 | 2 | 2147.91 | 0.09 | | | | | | |
|---|---|---|---|---|---|---|---|---|---|---|
| 2-5 | 722 | 15 | 3070.1 | 0.49 | 5.23 | (1.74-15.76) | 3.67 | (1.19-11.35) | 4.89 | (1.34-17.78) |
| >5 | 28 | 3 | 114.32 | 2.62 | 28.5 | (6.38-127.36) | 19.18 | (4.1-89.71) | 25.00 | (3.99-156.43) |

Abbreviations: HR, hazard ratio; CI, confidence interval; CVD, cardiovascular disease; IR, incidence rate; PY, person-years; Ref., reference category.
^{a}Score was based on methylation of 10 CpGs (cg01612140, cg05575921, cg06126421, cg08362785, cg10321156, cg14975410, cg19572487, cg23665802, cg24704287, cg25983901) using their respective first quartile values (cg08362785: using its highest quartile) among the ESTHER participants as the cutoff points to define aberrant methylation; Score 0-10 refer to simultaneously aberrant methylation at 0 to 10 CpGs;
^{b}Incidence rate per 100 person-years;
^{c}Model 1: without adjustment;
^{d}Model 2: adjusted for chronological age and sex.
^{f}Model 3: like model 2, additionally adjusted for smoking status, BMI, physical activity, alcohol consumption, systolic blood pressure, total cholesterol, hypertension, and prevalent cardiovascular disease, diabetes, and cancer at baseline.

TABLE 10
Associations of the risk score and epigenetic clock with all-cause and cause-specific
mortality in the ESTHER study

| | Mortality score^{a}/ | HR (95% CI) | | |
|---|---|---|---|---|
| Outcome | epigenetic clock^{b} | Model 1^{c} | Model 2^{d} | Model 3^{e} |
| All-cause mortality | 0 | Ref. | Ref. | Ref. |
| | 1 | 2.04 (1.11-3.75) | 2.02 (1.10-3.72) | 2.15 (1.09-4.21) |
| | 2-5 | 3.18 (1.81-5.59) | 3.07 (1.74-5.41) | 3.31 (1.75-6.28) |
| | >5 | 7.64 (4.21-13.85) | 7.18 (3.92-13.15) | 6.96 (3.46-14.01) |
| | Hannum Δage (per 5 years) | 1.27 (1.10-1.46) | 1.09 (0.94-1.27) | 1.08 (0.92-1.27) |
| Cancer mortality | 0 | Ref. | Ref. | Ref. |
| | 1 | | | |
| | 2-5 | 1.24 (0.72-2.11) | 1.19 (0.69-2.04) | 1.16 (0.65-2.06) |
| | >5 | 3.12 (1.69-5.78) | 2.89 (1.53-5.46) | 2.33 (1.12-4.84) |
| | Hannum Δage (per 5 years) | 1.25 (0.98-1.59) | 1.13 (0.87-1.46) | 1.15 (0.88-1.51) |
| CVD mortality | 0 | Ref. | Ref. | Ref. |
| | 1 | | | |
| | 2-5 | 3.41 (1.82-6.40) | 3.28 (1.74-6.18) | 3.85 (1.87-7.89) |
| | >5 | 7.19 (3.54-14.62) | 6.63 (3.19-13.78) | 8.47 (3.54-20.28) |
| | Hannum Δage (per 5 years) | 1.34 (1.05-1.71) | 1.12 (0.87-1.45) | 1.12 (0.85-1.48) |

Abbreviations:
HR, hazard ratio;
CI, confidence interval;
CVD, cardiovascular disease;
Ref., reference category.
^{a}Score was based on methylation of 10 CpGs (cg01612140, cg05575921, cg06126421, cg08362785, cg10321156, cg14975410, cg19572487, cg23665802, cg24704287, cg25983901) using their respective first quartile values (cg08362785: using its highest quartile) among the ESTHER participants as the cutoff points to define aberrant methylation; Score 0-10 refer to simultaneously aberrant methylation at 0 to 10 CpGs;
^{b}The epigenetic clock estimated by the difference between DNA methylation calculated according to Hannum's algorithm and chronological age;
^{c}Model1: adjusted for age and sex;
^{d}Model 2: like model 1, additionally adjusted for the epigenetic clock/risk score;
^{e}Model 3: like model 2, additionally adjusted for smoking status, BMI, physical activity, alcohol consumption, systolic blood pressure, total cholesterol, hypertension, and prevalent cardiovascular disease, diabetes, and cancer at baseline.

TABLE 11
Associations of the risk score and epigenetic clock (calculated according to Horvath's algorithm)
with all-cause and cause-specific mortality in the ESTHER study

| | Mortality score^{a}/ | HR (95% CI) | | |
|---|---|---|---|---|
| Outcome | epigenetic clock^{b} | Model 1^{c} | Model 2^{d} | Model 3^{e} |
| All-cause mortality | 0 | Ref. | Ref. | Ref. |
| | 1 | 2.04 (1.11-3.75) | 2.04 (1.11-3.75) | 2.18 (1.11-4.28) |
| | 2-5 | 3.18 (1.81-5.59) | 3.15 (1.78-5.55) | 3.45 (1.82-6.54) |
| | >5 | 7.64 (4.21-13.85) | 7.55 (4.14-13.76) | 7.47 (3.72-15.00) |
| | Horvath Δage (per 5 years) | 1.14 (1.00-1.30) | 1.02 (0.90-1.16) | 0.98 (0.85-1.13) |
| Cancer mortality | 0 | Ref. | Ref. | Ref. |
| | 1 | | | |
| | 2-5 | 1.24 (0.72-2.11) | 1.19 (0.69-2.05) | 1.19 (0.67-2.12) |
| | >5 | 3.12 (1.69-5.78) | 2.96 (1.58-5.54) | 2.49 (1.21-5.14) |
| | Horvath Δage (per 5 years) | 1.18 (0.95-1.47) | 1.10 (0.88-1.38) | 1.04 (0.82-1.32) |
| CVD mortality | 0 | Ref. | Ref. | Ref. |
| | 1 | | | |
| | 2-5 | 3.41 (1.82-6.40) | 3.40 (1.80-6.42) | 3.99 (1.94-8.20) |
| | >5 | 7.19 (3.54-14.62) | 7.17 (3.48-14.74) | 9.10 (3.81-21.71) |
| | Horvath Δage (per 5 years) | 1.15 (0.93-1.43) | 1.00 (0.81-1.25) | 1.00 (0.78-1.28) |

Abbreviations:
HR, hazard ratio;
CI, confidence interval;
CVD, cardiovascular disease;
Ref., reference category.
^{a}Score was based on methylation of 10 CpGs (cg01612140, cg05575921, cg06126421, cg08362785, cg10321156, cg14975410, cg19572487, cg23665802, cg24704287, cg25983901) using their respective first quartile values (cg08362785: using its highest quartile) among the ESTHER participants as the cutoff points to define aberrant methylation; Score 0-10 refer to simultaneously aberrant methylation at 0 to 10 CpGs;
^{b}The epigenetic clock estimated by the difference between DNA methylation calculated according to Horvath's algorithm¹³⁸ and chronological age;
^{c}Model 1: adjusted for age and sex;
^{d}Model 2: like model 1, additionally adjusted for the epigenetic clock/risk score;
^{e}Model 3: like model 2, additionally adjusted for smoking status, BMI, physical activity, alcohol consumption, systolic blood pressure, total cholesterol, hypertension, and prevalent cardiovascular disease, diabetes, and cancer at baseline.

1. A method for determining a survival probability of a subject comprising
a) detecting the methylation status of at least two CpG sites selected from the list consisting of cg24704287, cg08362785, cg25983901, cg06126421, cg05575921, cg23665802, cg01612140, cg19572487, cg14975410, and cg10321156 in a sample of said subject and,
b) based on the methylation status detected in step a), determining the survival probability of said subject.

2. The method of claim 1, wherein the methylation status of at least three, of said CpG sites is detected.

3. The method of claim 1, wherein the methylation status of all ten methylation sites is determined.

4. The method of claim 1, wherein said detecting the methylation status of a CpG site is detecting the average degree of methylation of said site from at least 10.

5. The method of claim 1, to wherein said method comprises further step a1) comparing the methylation status of said at least two CpG sites of step a) to references; and wherein in step b) the determining is based on the comparison of step a1).

6. The method of claim 4, wherein said reference value or reference range is an average degree of methylation of ≤0.34 for cg01612140; ≤0.78 for cg05575921; ≤0.60 for cg06126421; ≥0.67 for cg08362785; ≥0.39 for cg10321156; ≤0.45 for cg14975410; ≤0.49 for cg19572487; ≤0.30 for cg23665802; ≤0.31 for cg24704287; and/or ≤0.49 for cg25983901.

7. The method of claim 1, wherein said sample is a bodily fluid sample.

8. The method of claim 1, wherein an unfavorable survival probability is determined if a methylation status deviating from the reference is detected.

9. The method of claim 1, wherein determining said survival probability comprises determining a mortality risk.

10. (canceled)

11. (canceled)

12. A data collection, comprised on a data carrier, comprising the positions of at least two, CpG sites selected from cg24704287, cg08362785, cg25983901, cg06126421, cg05575921, cg23665802, cg01612140, cg19572487, cg14975410, and cg10321156.

13. A kit comprising means for determining the methylation status of at least two CpG sites selected from the CpG sites cg24704287, cg08362785, cg25983901, cg06126421, cg05575921, cg23665802, cg01612140, cg19572487, cg14975410, and cg10321156, and a data collection according to claim 12.

14. A device comprising an analysis unit comprising means for determining the methylation status of at least two CpG sites selected from cg24704287, cg08362785, cg25983901, cg06126421, cg05575921, cg23665802, cg01612140, cg19572487, cg14975410, and cg10321156, and an evaluation unit comprising a data collection according to claim 12.

15. The method of claim 1, further comprising the step of providing close monitoring and/or lifestyle recommendations in case an unfavorable survival probability and/or an increased mortality risk is detected.

16. The method of claim 1, wherein an unfavorable survival probability is determined if a methylation status deviating from the reference is detected for at least two CpG sites.

17. The method of claim 1, wherein an unfavorable survival probability is determined if a methylation status deviating from the reference is detected for at least five CpG sites.
